(19) United States
(12) Patent Application Publication
(10) Pub. No.: US 2007/0185489 A1
(43) Pub. Date: Aug. 9, 2007

(54) Devices and Methods for Inter-Vertebral Orthopedic Device Placement

(76) Inventor: M. S. Abdou, San Diego, CA (US)

Correspondence Address: FISH & RICHARDSON, PC, P.O. BOX 1022, MINNEAPOLIS, MN, 55440-1022, US

(21) Appl. No.: 11/627,910

(22) Filed: Jan. 26, 2007

Related U.S. Application Data
(60) Provisional application No. 60/763,047, filed on Jan. 26, 2006.
(60) Provisional application No. 60/800,959, filed on May 17, 2006.

(51) Int. Cl. A61F 2/30

(52) U.S. Cl. 606/61; 623/17.11

(57) ABSTRACT

A spinal fixation device is described. The device can safely promote fusion across one or more vertebral fusing levels while simultaneously supporting vertebral motion at other levels.

### REFERENCE TO PRIORITY DOCUMENT

This application claims priority of the following co-pending U.S. Provisional Patent Applications: (1) U.S. Provisional Patent Application Ser. No. 60/763,047, filed Jan. 26, 2006; and (2) U.S. Provisional Patent Application Ser. No. 60/800,959, filed May 17, 2006. Priority of the aforementioned filing dates is hereby claimed and the disclosure of the Provisional Patent Applications are hereby incorporated by reference in their entirety.

### BACKGROUND

Spinal disease is a major health problem in the industrialized world and the surgical treatment of spinal pathology is an evolving discipline. Currently, resection of the painful disc and fusion of the adjacent vertebral bodies has emerged as the most common surgical treatment of degenerative disc disease.

The growing experience with spinal fusion has shed light on the long-term consequences of vertebral immobilization. It is now accepted that fusion of a specific spinal level will increase the load on the spinal segments immediately above and below the fused level. Further, as a consequence of fusion, each adjacent disc will experience a displaced center of rotation and produce an aberrant motion profile. The increased load and abnormal movement experienced by the adjacent discs will synergistically act to accelerate the rate of degeneration at these levels. Consequently, the number of patients who require extension of their fusion to the adjacent, degenerating levels has increased with time.

In the cervical spine, many individuals have degenerative changes of varying severity at multiple discs. When pain, weakness and other symptoms arise, it is not uncommon to find that the symptomatic disc is surrounded by diseased but less-degenerated adjacent disc levels. Unfortunately, resection and fusion of the symptomatic disc will increase the load on the adjacent segments and accelerate the rate of degeneration at those levels. With time, the adjacent disc levels will also require resection and fusion. The second procedure necessitates re-dissection through the prior, scarred operative field and carries a greater risk of complications than the initial procedure. Further, extension of the fusion will increase the load on the motion segments that now lie at either end of the fusion construct and will accelerate the rate of degeneration at those levels. Thus, spinal fusion begets additional, future fusion surgery.

It would be advantageous to treat the symptomatic level while minimizing the negative biomechanical consequences of fusion on the adjacent disc levels. Clearly, a device that can promote fusion at the desired level(s) while maintaining and supporting vertebral motion at other level(s) is needed. U.S. Pat. Nos. 6,293,949 and 6,761,719 illustrate a method of dynamic vertebral stabilization. In that invention, bone screws are placed into each of two vertebral bodies and a malleable member is used to connect them. The malleable member dampens movement between the vertebral bodies and returns the vertebrae to the neutral position after the force acting upon the construct has dissipated.

Unfortunately, the devices illustrated cannot accommodate vertebral fusion. During fusion, bone re-absorption at the bone/graft interface is the first step in the healing process. After re-absorption, the fusing bones must settle and reestablish contact with one another in order for the fusion to progress. Since the devices illustrated in the referenced patents are designed to return the vertebrae to the neutral position, they will actively oppose bone settling and forcefully separate the vertebral bodies as they try to re-establish bony contact. Thus, placement of these devices across a disc level that is to be fused would inhibit bone healing, preclude formation of the fusion mass and insure failure of the bony fusion.

The vertebral bodies immediately adjacent to a fused disc space will exhibit abnormal motion characteristic and this motion profile will accelerate the degenerative process. The disc space above the fused level, for example, will experience a downward migration of the center of rotation so that the upper vertebral body will follow a substantially spherical path of greater radius (i.e., lesser curvature) in the sagittal (anterior-posterior) plane relative to the lower body. The alteration in trajectory will produce greater translational movement of the upper vertebral body in the anterior-posterior plane and subject the intervening disc to a significant increase in shear forces. The devices illustrated in the referenced patents do not correct the aberrant motion seen adjacent to a fused segment and, in fact, make no attempt to favor any particular motion pattern. Since the devices are attached to bone at each end and have an intervening malleable member of uniform design and resistance, it is impossible for them to simultaneously support the widely divergent motion requirements of a fusion at one level and a mobile segment at another level.

The referenced devices have a bellows-like design that may entrap, pinch and injure the surrounding soft tissues within the expanding and contracting folds of the moving implant. The use of super-elastic materials for device manufacture will only add to the extent of travel and further risk tissue entrapment. Since the device is placed onto the anterior aspect of the cervical spine, it is positioned immediately adjacent to the esophagus and the pharynx and may injure these structures with movement. The vast experience gained with bone plate fixation of this region has unequivocally shown that injury of the pharynx and/or esophagus is among the most feared surgical complications. Should injury occur, serious infection with significant risk of long term morbidity or even mortality will almost certainly develop. Further, the malleable member may fracture with repetitive movement. With failure, these devices can fragment and produce sharp subsegments that can injure the critical tissues contained within the intended area of implantation. In short, the placement of an uncontained bellows-like mechanism immediately behind these critical soft-tissue structures is dangerous.

### SUMMARY

There remains a need in the art for a device that can safely promote fusion across one or more fusing levels while simultaneously supporting vertebral motion at other levels.

In one embodiment, a hybrid fixation device is illustrated. In one segment, the device is adapted to span and accommodate a fusing segment while at another segment the device has a malleable member that supports vertebral motion. The malleable segments is contained within a biocompatible sheath or membrane that serves to contain implant ware debris, keep the soft tissue out of the mobile implant sub-segments, contain implant fragments in case of failure and, if desired, allow placement and containment of a biocompatible lubricant. Multiple embodiments of the fusion fixation segment are provided. A modular device is also provided.

In an additional embodiment, a multi-segmental device that limits vertebral motion to a spherical path is illustrated. The device can be used to define the center of rotation and correct aberrant motion patterns. In another embodiment, the device is fitted with a malleable member so that it can support vertebral motion. Additional versions illustrate the addition of an intra-disc attachment that can also define the center of rotation for the motion segment. Multiple embodiments are illustrated where the motion of the attached vertebral bodies is supported by malleable members of various designs. Any of the disclosed dynamic implants may be coupled to segments that accommodate fusion so that the hybrid assembly can support fusion at one level and dynamic motion at another level. Finally, the addition of a biocompatible sheath or membrane is illustrated for a rod-based dynamic stabilization implant.

The implants described in this application can safely promote fusion across one or more fusing levels while simultaneously supporting vertebral motion at other levels. Other features and advantages will be apparent from the following description of various embodiments, which illustrate, by way of example, the principles of the disclosed devices and methods.

### BRIEF DESCRIPTION OF THE DRAWINGS

FIG. 1 is a perspective view of a dynamic bone fixation device configured to retain bone portions such as vertebra of a spinal column in a desired spatial relationship.

FIG. 2A shows an assembled view of the device.

FIG. 2B shows an exploded view of the bone fixation device.

FIG. 3 shows the device attached to the vertebrae V1, V2, and V3.

FIGS. 4 and 5 show top plan views of alternate embodiments the device that permits limited movement between the vertebrae V2 and V3.

FIGS. 6 and 7 show another alternate embodiment of the device.

FIG. 8 shows another embodiment of a bone fixation device in an assembled state and attached to a pair of vertebrae V1 and V2.

FIG. 9 shows a first perspective view of the device of FIG. 8 in an exploded state.

FIG. 10 shows a second perspective view of the device of FIG. 8 in an exploded state.

FIG. 11 shows a cross-sectional view of the assembled device attached to the vertebrae V1 and V2.

FIG. 12 shows another embodiment of a dynamic bone fixation device in an assembled state and attached to a pair of vertebrae V1 and V2.

FIG. 13 shows a first perspective view of the device of FIG. 12 in an exploded state.

FIG. 14 shows a perspective view of the device of FIG. 12 in an exploded state.

FIG. 15 shows various view of a malleable member 1420.

FIGS. 16a-16c show alternate embodiments of the device.

FIGS. 17 and 18 show perspective and cross-sectional view of an embodiment of the device.

FIGS. 19-20 show perspective and cross-sectional views of an embodiment of the device that is similar to the device shown in FIGS. 17 and 18.

FIG. 21 shows a perspective view of an embodiment of a dynamic bone fixation device in an assembled state and attached to a pair of vertebrae V2 and V3.

FIG. 22 shows an exploded view of the device of FIG. 21.

FIGS. 23 and 24 show the device having different embodiments of the plate member that permits movement between the vertebrae V2 and V3.

FIG. 25 shows a perspective view of an embodiment of a dynamic bone fixation device in an assembled state and attached to a pair of vertebrae V1 and V2.

FIG. 26 shows a perspective view of the device of FIG. 25 in an exploded state.

FIG. 27 shows various views of the bone screw receiver.

FIGS. 28 and 29 show perspective and cross-sectional views of the device of FIG. 25.

FIGS. 30 and 31 show another embodiment of the device of FIG. 25.

FIG. 32 shows a perspective, assembled view of another embodiment of a dynamic: bone fixation device.

FIG. 33 shows an exploded view of the device of FIG. 32.

FIG. 34 shows various views of an articulating member that movably links the plate components of the device.

FIG. 35 shows a cross-sectional view of the device of FIG. 32 attached to vertebrae.

FIG. 36 shows another embodiment of a bone fixation device.

FIG. 37 shows an enlarged view of a receiver and coupler of the device.

FIG. 38 shows various views of the coupler and receiver in an assembled state.

FIGS. 39 and 40 show top and bottom perspective views of another embodiment of a device.

FIG. 41 shows a top view of the device of FIG. 39.

FIG. 42 shows yet another embodiment of a bone fixation device.

FIG. 43 shows an exploded view of the device of FIG. 42.

FIG. 43a illustrates a partial view of another embodiment of a dynamic bone fixation device.

FIG. 44 is a perspective view of another embodiment of a dynamic bone fixation device.

FIG. 45 shows the device of FIG. 44 in an exploded state.

FIGS. 46 and 47 show the device of FIG. 44 attached to vertebrae V1 and V2.

### DETAILED DESCRIPTION

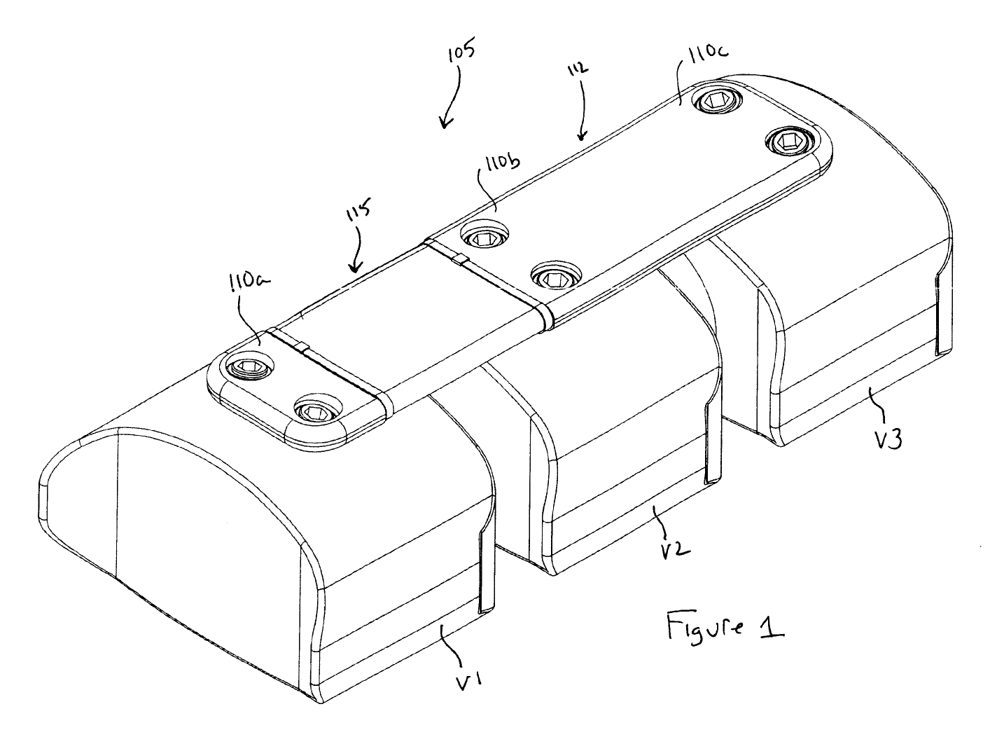

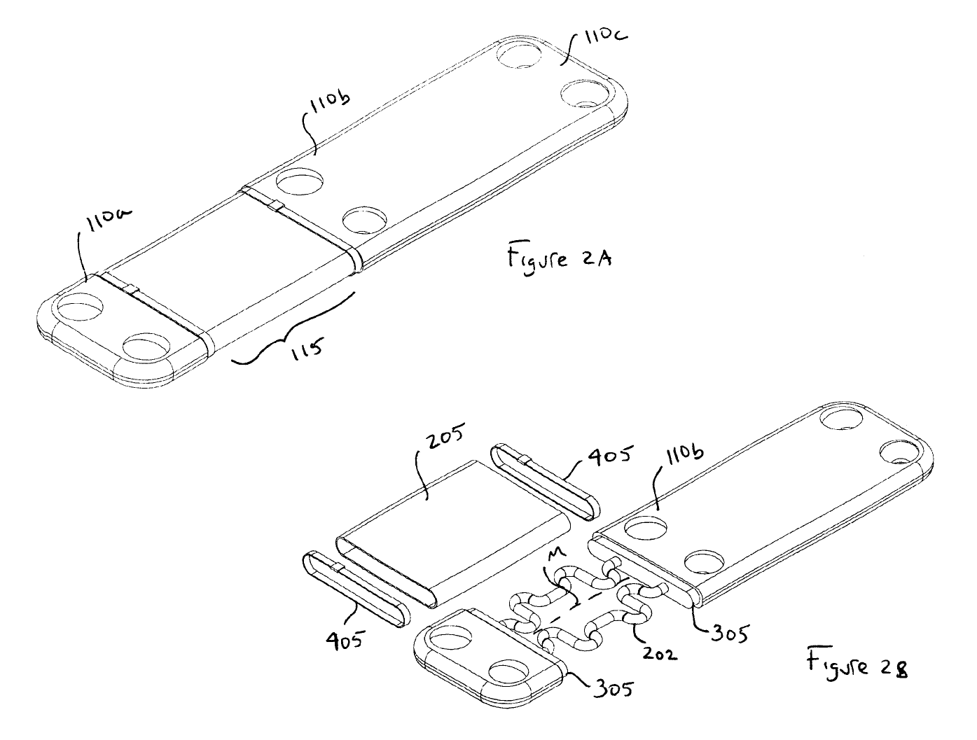

FIG. 1 is a perspective view of a dynamic bone fixation device 105 configured to retain bone portions such as vertebra of a spinal column in a desired spatial relationship. FIG. 1 shows the device 105 attached to three vertebrae V1, V2, and V3. FIG. 2A shows an assembled view of the device and FIG. 2B shows an exploded view of the bone fixation device 105. With reference to FIGS. 1-2B, the device 105 includes a first connection portion 110a malleably linked to al plate 112 that includes connection portions 110a and 110b. The connection portions are adapted to attach to respective vertebrae, such as via bone screws, or other fasteners, that are positioned through boreholes. The device 105 also includes at least one malleable portion 115 that malleably links the connection portions 110a to the plate 112. The connection portions 110 and malleable portion 115 shown in FIGS. 1 and 2 are exemplary and it should be appreciated that the portions can have other shapes and structures that are adapted to be attached to vertebrae and permit relative motion therebetween. In one embodiment, the device is curved to conform to the surface contour of the vertebral bodies at the site of implantation. In the cervical spine, for example, the posterior surface of the device that abuts the anterior surface of the vertebral bodies can be concave in both the longitudinal and horizontal planes.

With reference to FIG. 2B, the device 105 includes attachment structures such as annular seats 305 that enable a sheath 205 to be attached thereto. In this regard, the sheath 205 mates with the annular seats 305. The device 105 can include clamps 405 that encircle the seats 305 and exert a compressive force to removably fix the sheath 205 to the device 105. Other structures or mechanisms can be used to attach the sheath 205 to the device 105.

With reference still to FIG. 2B, the malleable portion 115 includes at least one malleable member 202 covered by a malleable sheath 205. The malleable member 202 and sheath 205 are adapted to alter in shape so as to permit at least limited, relative displacement between the connection members 110a and the plate 112. The malleable member 202 can be an undulated rod that loops back and forth about a longitudinal axis of the device 105. The undulating shape permits the malleable member 202 to flex, move or otherwise change shape along a direction parallel to the midline M and/or a direction transverse or cross-wise to the midline M. It should be appreciated that the malleable member 202 can have any of a variety of shapes that are adapted to be malleable. In the illustrated embodiment, the malleable members 202 are integral with the plate 112 and connection portion 110a so that they form a unitary structure or construct. However, the connection portion and the plate can be formed separate from the malleable members 202 and attached by any method known to one of ordinary skill in the art, such as, for example, by fastening or welding.

The sheath 205 encloses or encapsulates respective malleable member(s) 202. The sheath 205 is sized relative to the malleable member 202 such that there is sufficient space to permit movement of the malleable member 202 inside the sheath 205. The sheath 205 functions to contain shed particulate debris and keep adjacent tissues and scar out of the mobile core and, if desired, permit placement of a biocompatible lubricant within the space 505. Should the malleable member 202 fail and fracture, the sheath 205 would also serve to contain the fragments and keep the device ends attached to one another.

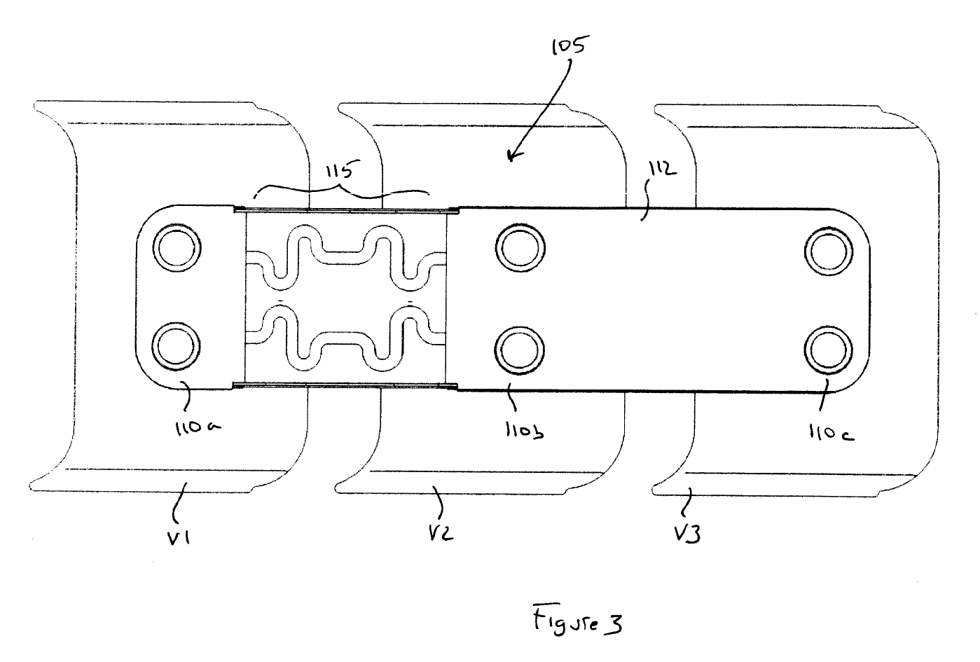

For the embodiment shown in FIGS. 1 and 2A/2B, each of the connection portions 110 is adapted to be attached to a respective vertebra. This is accomplished by inserting at least one bone screw through a borehole in the connection portion and fixating the bone screw and connection portion to the vertebra. FIG. 3 shows the device 105 attached to the vertebrae V1, V2, and V3. The device 105 can have various structural shapes and configurations. For example, the device can include a central aperture that provides access to the disc space between the vertebrae V2 and V3 (as shown in FIG. 5). The device can also include a slot that provides a location where a distraction screw can be attached to the underlying vertebra (as shown in FIG. 5).

Each borehole 110c that overlies vertebra V3 has a largely spherical configuration and a bottom aperture that has a diameter greater than that of the bone screws. Because of the size differential between the bottom aperture of the borehole and the diameter of the screw, the spherical head of the screw can rotate within the borehole. This mechanism permits movement of vertebra V3 towards vertebra V2. Conversely, each borehole 110a and 110b has a bottom aperture with a diameter minimally larger than that of the bone screw so that, once seated, the screws are constrained and relatively immobile within the plate. Movement between vertebrae V1 and V2 is provided by the action of the malleable portion 115. In this way, the device allows vertebra V1 to move from a first position to a second position relative to vertebra V2 in reaction to an applied force and then to substantially return to the first position when the force has dissipated. When attached to the vertebrae V1, V2 and V3, this hybrid device will fixate vertebrae V2 and V3 relative to one another so as to promote fusion at this level while malleable portion 115 will support motion at the non-fused level between vertebrae V1 and V2. As an alternative embodiment, each borehole 110c has a bottom aperture with a diameter minimally larger than that of the bone screw so that, once seated, the screws are constrained and relatively immobile within the plate. In this way, the device accommodates fusion by rigidly affixing vertebra V2 to vertebra V3.

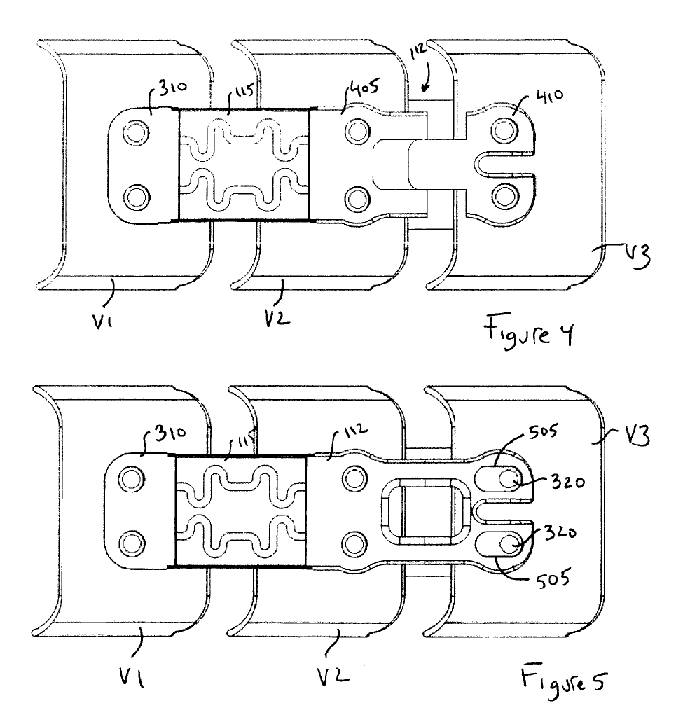

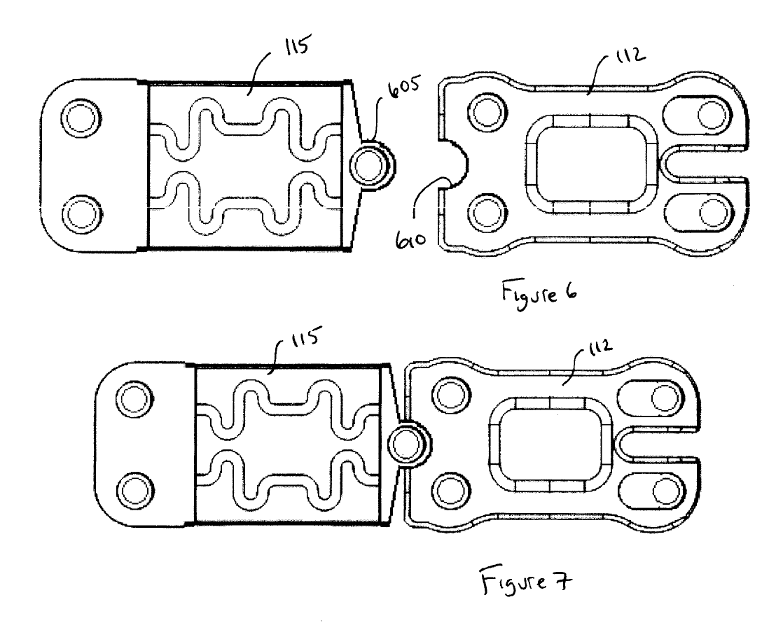

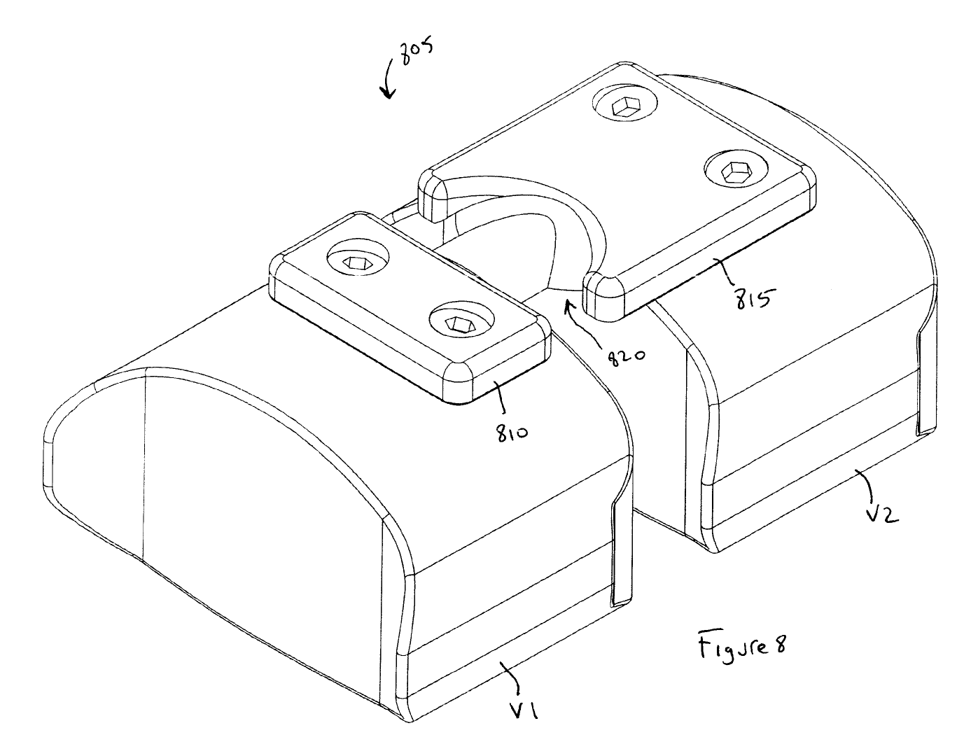

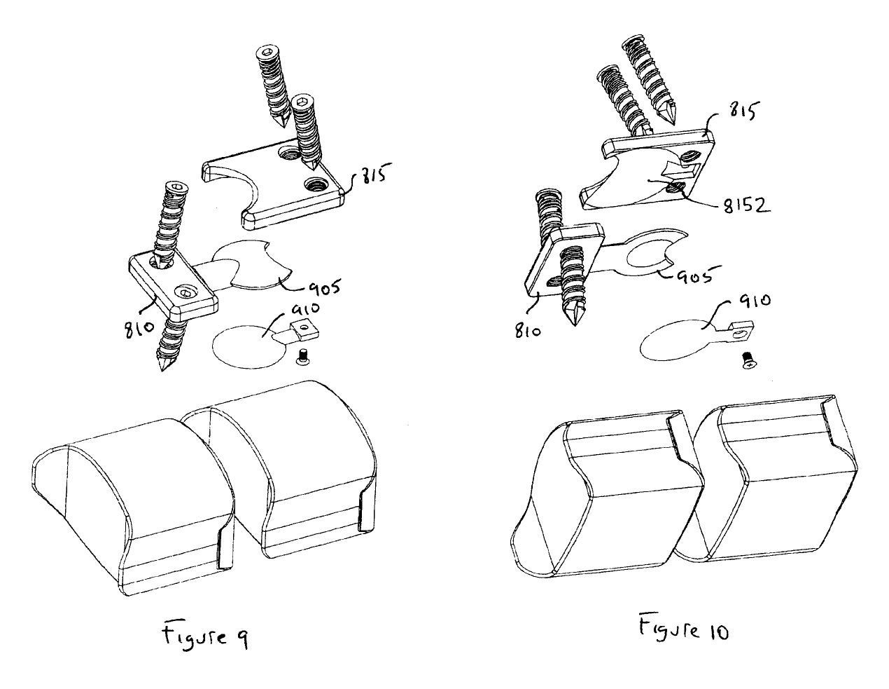

FIGS. 4 and 5 illustrate top plan views of alternate embodiments. The devices include a first connection portion 310 and a malleable portion 115 that are similar to that described with reference to FIG. 3 and a modified plate portion 112. When fully seated, the bone screws at all levels provide no significant movement between the screw and the device's boreholes. With reference to the embodiment of FIG. 4, the plate 112 is formed of two components 405 and 410 that are movably coupled to one another. Component 405 includes a slot that slidably receives a tongue of the component 410 and the tongue slides along the slot so as to permit movement between the vertebrae V2 and V3. Movement between vertebrae V1 and V2, however, is provided by the action of the malleable portion 115. In this way, the device allows vertebra V1 to move from a first position to a second position relative to vertebra V2 in reaction to an applied force and then to return to the first position when the force acting upon the vertebrae has dissipated. The device also allows unopposed boney subsidence between vertebrae V2 and V3.

With reference now to the embodiment of FIG. 5, the plate 112 includes a pair of bore holes 505 for receipt of bone screws 320 that attach the plate 315 to the vertebra V3. The boreholes 505 are oversized relative to the size of the heads of the bone screws 320. In one embodiment, the boreholes 505 are elongated such that the boreholes 505 are slot-like. The relative size between the screw heads and the boreholes 505 permits the plate 112 to move relative to the bone screws 320 along the length of the boreholes and thereby permits unhindered boney subsidence between the V2 and V3 vertebrae. As before, movement between vertebrae V1 and V2 is provided by the action of the malleable portion 115.

FIGS. 6 and 7 illustrate an alternate embodiment of the device 105. In this embodiment, the plate 112 is formed of a single plate member with elongated boreholes 505 as in the embodiment of FIG. 5. The malleable portion 115 includes a coupler 605 that removably mates with a complementary-shaped coupler 610 on the plate 112. The coupler 605 comprises a protrusion and the coupler 610 comprises a cavity that is sized and shaped to lockingly receive the protrusion. In this manner, the couplers 605 and 610 can be mated to modularly attach the malleable portion 115 to the plate 112. While the fusion portion of the embodiment in FIG. 5 is illustrated, it should be appreciated that the configuration of either FIG. 3 or FIG. 4 could be alternatively used. Further, alternative modular couplers are known in the art and could be alternatively employed. U.S. Pat. Nos. 6,645,208; D505,205 and Pub. No. 2003/0074001 illustrate some of these alterative coupling methods.

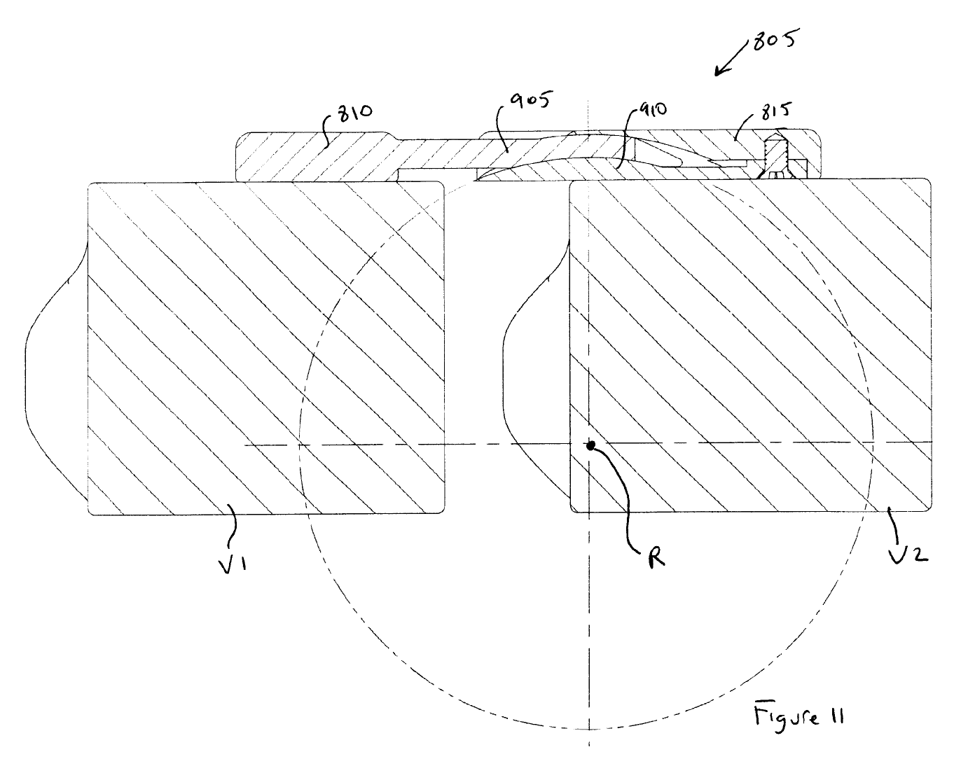

FIG. 8 shows another embodiment of a dynamic bone fixation device 805 in an assembled state and attached to a pair of vertebrae V1 and V2. FIG. 9 shows a first perspective view of the device of FIG. 8 in an exploded state. FIG. 10 shows a second perspective view of the device of FIG. 8 in an exploded state. The device 805 includes a first connection member 810 that attaches to the first vertebra V1 and a second connection member 815 that attaches to the second vertebra V2. The connection members are fixed to the respective vertebrae using bone screws that extend through boreholes in the connection members. An articulation assembly 820 interconnects the first and second connection members to permit relative movement therebetween, as described below. The relative movement can follow the contour of a sphere or globe. While a threaded screw/borehole configuration is shown as the one embodiment, it should be appreciated that any appropriate screw/borehole/device-to-screw locking mechanism may be alternatively employed.

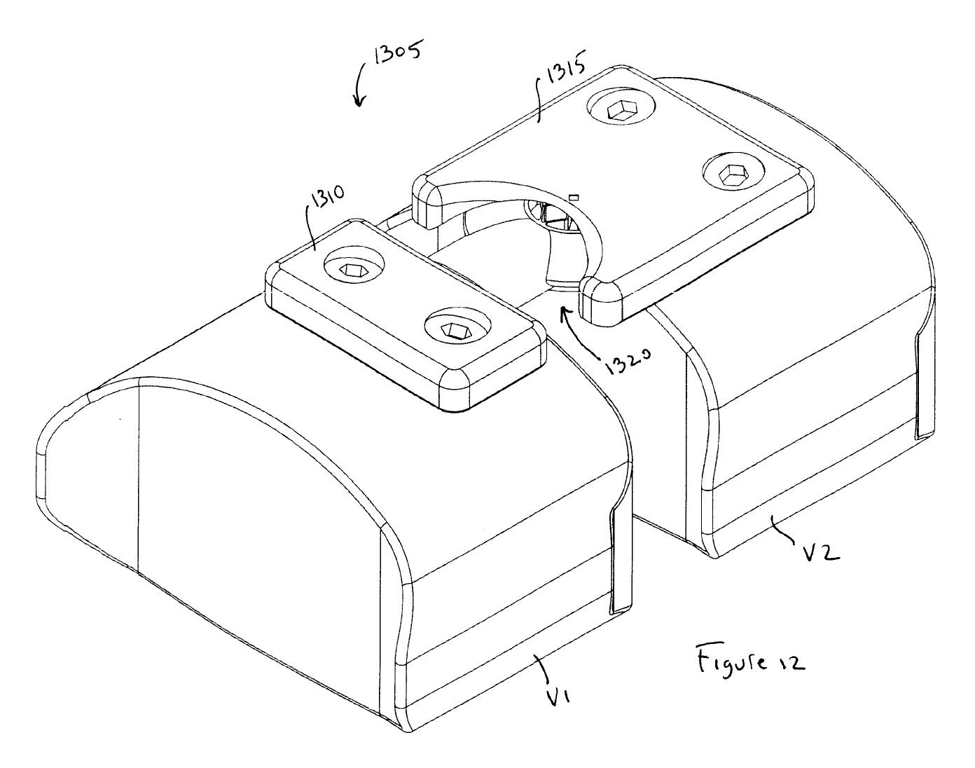

With reference to FIGS. 9 and 10, the articulation assembly 820 includes a first articulation member 905 that extends outwardly from the first connection member 810. The first articulation member 905 movably mates with a second articulation member 910 and the inferior surface 8152 of member 815. The articulation surfaces include complementary dome-shaped portions that couple to one another and have a common center of rotation. This is described in more detail with reference to FIG. 11, which shows a cross-sectional view of the assembled device 805 attached to the vertebrae V1 and V2. The first articulation member 905 has upper and lower surfaces that are dome-shaped (i.e., sphere-shaped). First articulation member 905 sits between member 815 and second articulation member 910. The upper surface of member 905 articulates with the complementary lower surface of member 815 while the lower surface of member 905 articulates with the complementary dome-shaped upper surface of the second articulation member 910. In an embodiment, the dome-shaped surfaces are defined by radii of curvature that originate at a common point R (FIG. 11), which corresponds to the physiological center of rotation (also referred to as the Instantaneous Axis of Rotation (IAR)) between vertebrae V1 and V2. The complementary shapes of the domed surfaces permit members 810 and 815 to move relative to one another and follow a physiological trajectory that is defined by the common center point. In this manner, the device 805 permits physiological movement between the vertebrae VI and V2 while fixating the vertebrae to one another. The assembled device 805 will permit unhindered boney subsidence and can be used as a fixation device at the fusion level.

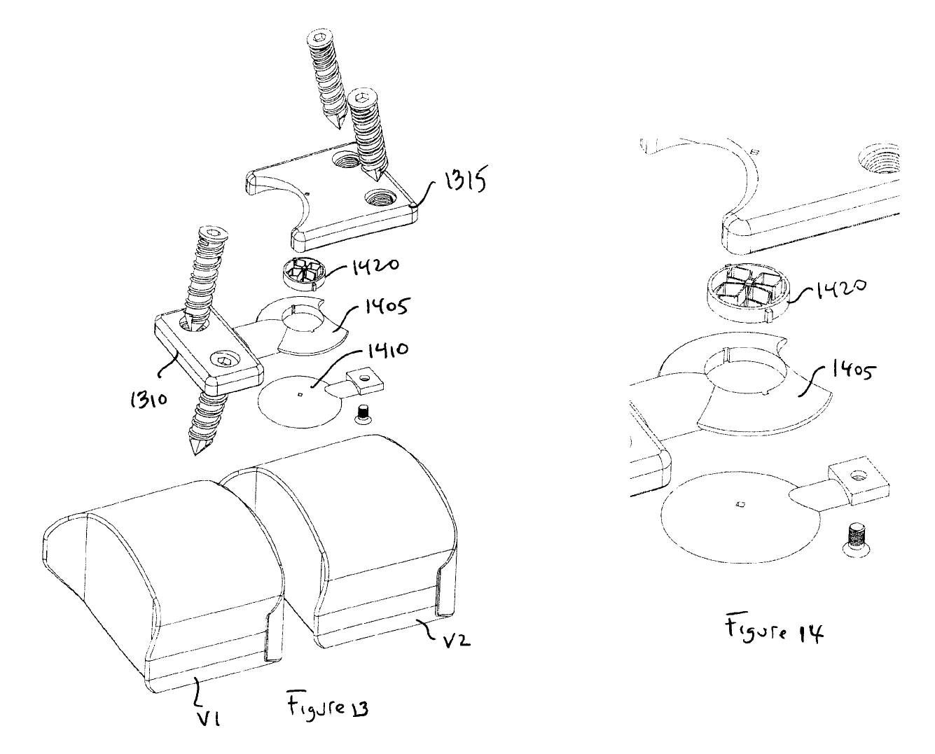

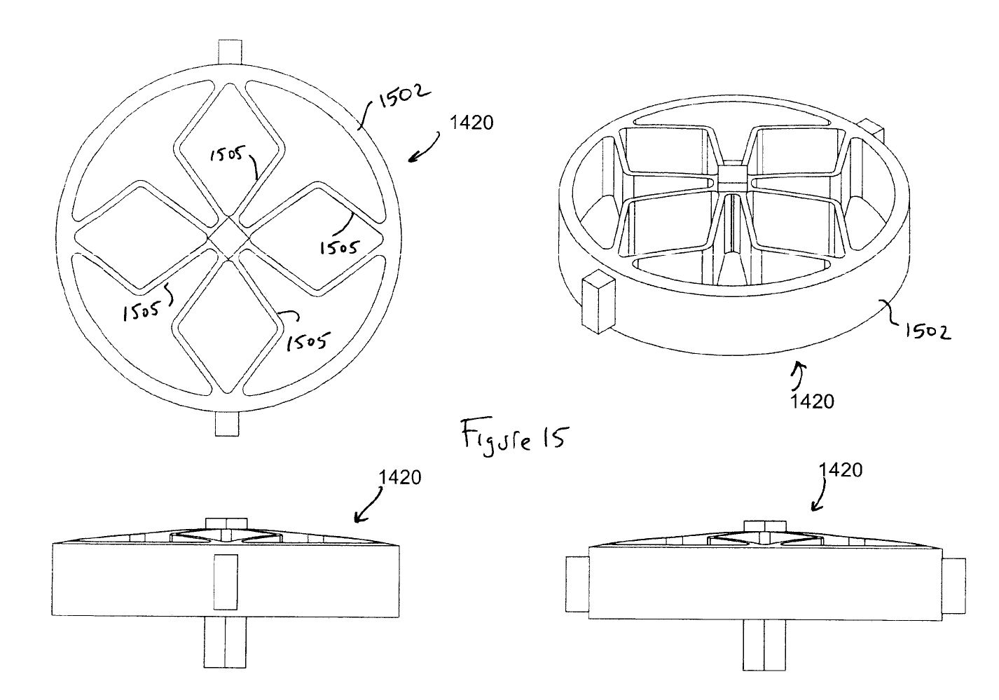

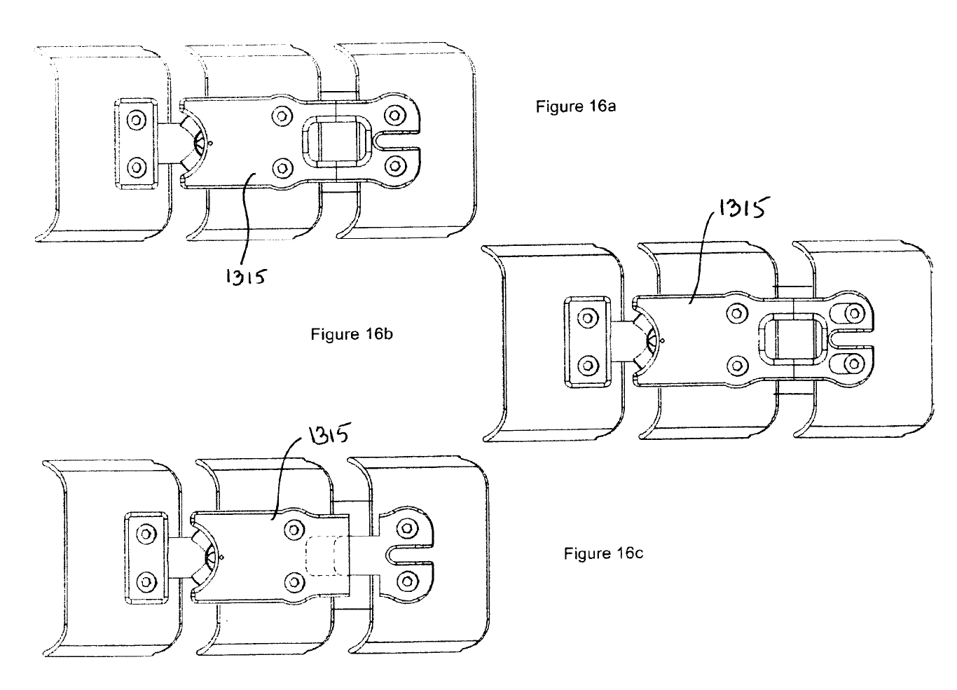

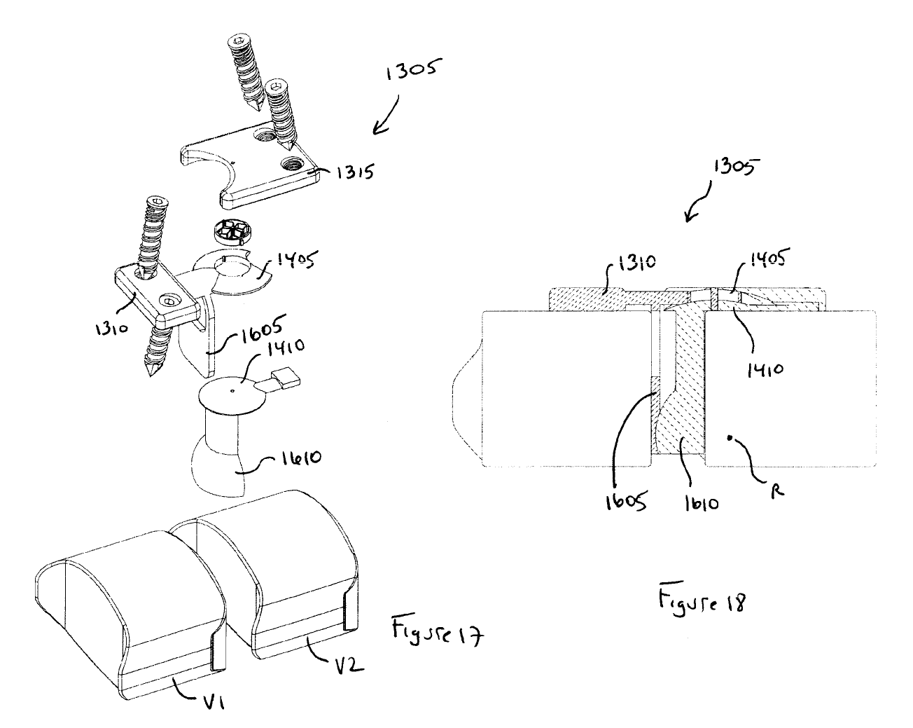

FIG. 12 shows another embodiment of a dynamic bone fixation device 1305 in an assembled state and attached to a pair of vertebrae V1 and V2. FIG. 13 shows a first perspective view of the device of FIG. 12 in an exploded state. FIG. 14 shows a second perspective view of the device of FIG. 12 in an exploded state. The device 1305 includes a first connection member 1310 that attaches to the first vertebra V1 and a second connection member 1315 that attaches to the second vertebra V2. The connection members are fixed to the respective vertebrae using bone screws that extend through boreholes in the connection members. An articulation assembly 1320 interconnects the first and second connection members to permit relative movement therebetween, as described below.

With reference to FIGS. 13 and 14, the articulation assembly 1320 includes a first articulation member 1405 that extends outwardly from the first connection member 1310. The lower surface of articulation member 1405 movably mates with the upper surface of second articulation member 1410 while the upper surface of member 1405 articulates with the spherical lower surface of member 1315. The articulation members are movably coupled to one another and travel in a spherical trajectory about a common center point as described above for the previous embodiment. In addition, a malleable member 1420 is located within a cavity inside the first articulation member 1405.

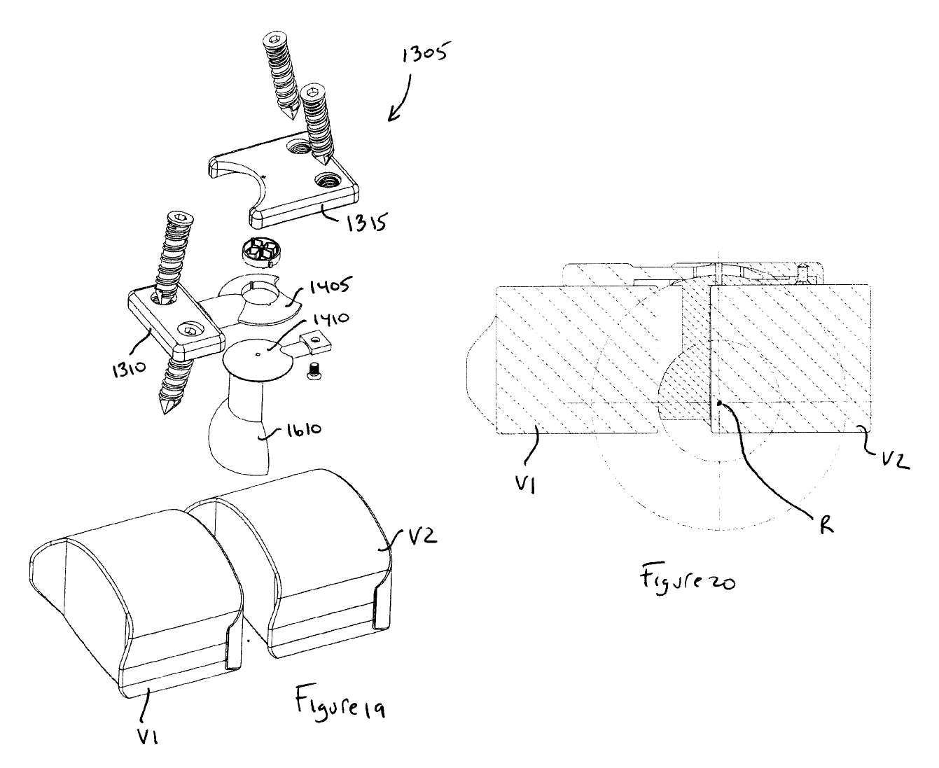

FIG. 15 shows various view of the malleable member 1420. Member 1420 includes an annular outer member 1502 that surrounds a series of diamond-shaped cells 1505. The diamond shape of the cells 1505 permit the cells to flex or otherwise change shape in response to a force applied to outer member 1502. The cells are biased toward a default shape so that the malleable member 1420 is also biased toward the default shape shown in FIG. 15 and member 1420 will return to the default shape after a force acting upon it has dissipated. In this manner, the device 1305 (FIG. 12) allows vertebra V1 to move from a first position to a second position relative to vertebra V2 in reaction to an applied motive force and then to return to the first position when the force acting upon the vertebrae has dissipated. Since the device restricts vertebral motion to that allowed by the spherical articulating surfaces, it also corrects aberrant vertebral movement and re-establishes a more physiological motion profile. A hybrid device that employs one or more of the current device embodiments at the non-fusion levels and one or more of the preceding device embodiments at the fusion levels can be used to accommodate both fusion and dynamic movement at different disc levels. As illustrated in FIGS. 16a-16c, the fusion device embodiments shown in FIGS. 3, 4, and 5 may be alternatively used with the current embodiment shown in FIG. 12 to produce additional hybrid device embodiments.

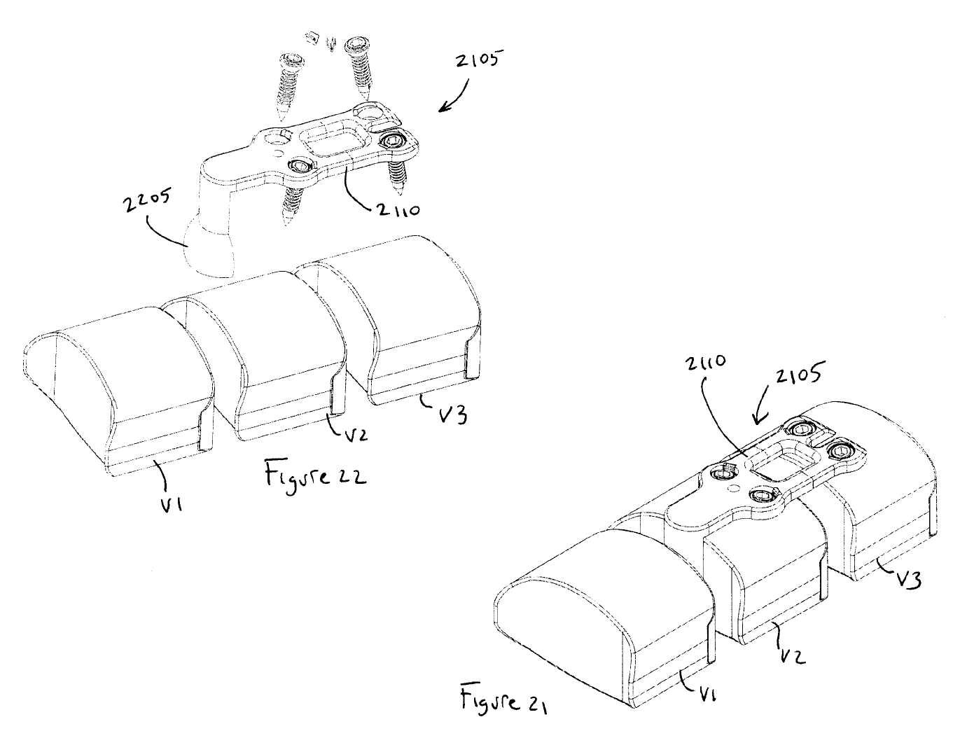

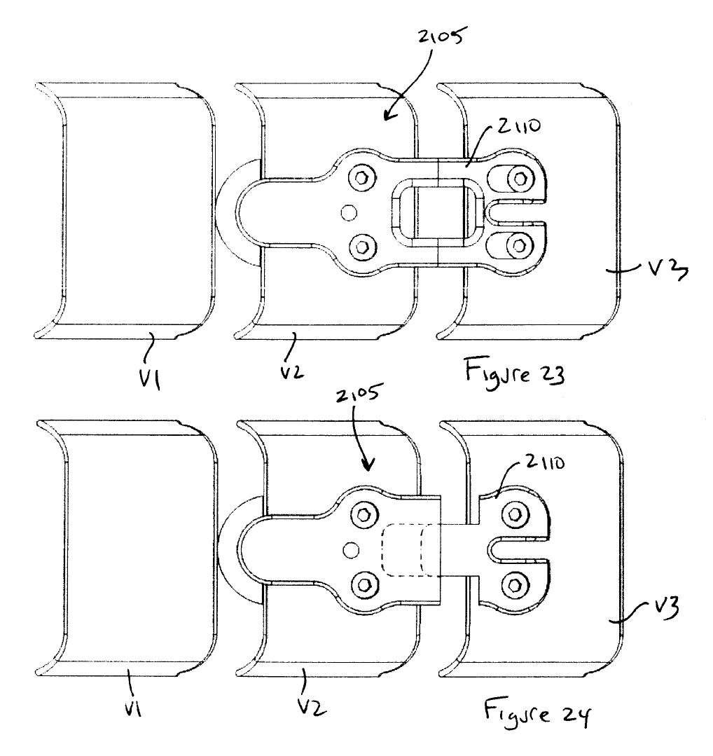

FIGS. 17 and 18 show perspective and cross-sectional views of an embodiment of the device 1305 that also includes articulation members 1605 and 1610 that are positioned within the disc space between the vertebrae V1 and V2. The articulation member 1605 extends outward from the first connection member 1310 into the disc space while the articulation member 1610 extends outward from the second articulation member 1410 into the disc space. The articulation members 1605 and 1610 include complementary domed surfaces that permit rotational movement therebetween.

As in the previous embodiment, the dome-shaped surfaces of the articulation members 1405, 1410, 1605, 1610 are defined by radii of curvature that originate at a common point R (FIG. 18), which can correspond to the physiological center of rotation between vertebrae V1 and V2. The complementary shapes of the domed surfaces permit the articulation members to move relative to one another in a spherical trajectory. In this manner, the device 805 permits physiological rotational movement between the vertebrae V1 and V2 while securing the vertebrae to one another.

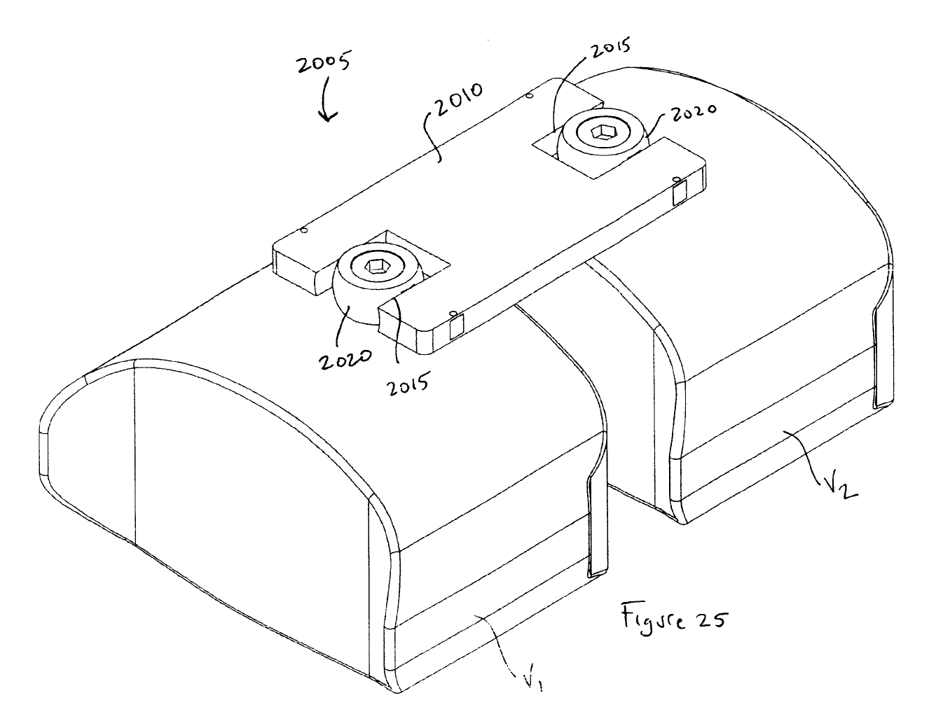

FIGS. 19 and 20 show perspective and cross-sectional views of an embodiment of the device 1305 that is similar to the device shown in FIGS. 17 and 18. However, the embodiment shown in FIGS. 19 and 20 does not include the articulation member 1605. Rather, the articulation member 1610 extends outward from the second articulation member 1410 into the disc space such that the domed surface directly abuts the vertebra V1, as shown in FIG. 20. As in the previous embodiment, the dome-shaped surfaces of the articulation members are defined by radii of curvature that originate at a common point R (FIG. 20), which corresponds to the physiological center of rotation of the vertebrae V1 and V2.

As alternative embodiments, the articulating surfaces of the embodiment in FIGS. 8 to 11 and/or the embodiments in FIGS. 12 to 20 may be altered so as to produce a device with a variable center of rotation. This can be produced most easily by producing a “loose” articulation at each of the upper and lower bearing surfaces of these two members. In an embodiment, the loose articulation is created by slightly increasing the radius of the bearing surface that contacts and interacts with the superior bearing surfaces of members 905/1405 while also decreasing the radius of the bearing surface that contacts and interacts with the inferior surfaces of members 905/1405. The variable center of rotation can be similarly created by a multitude of other member modifications that would be apparent to one of ordinary skill in the art.

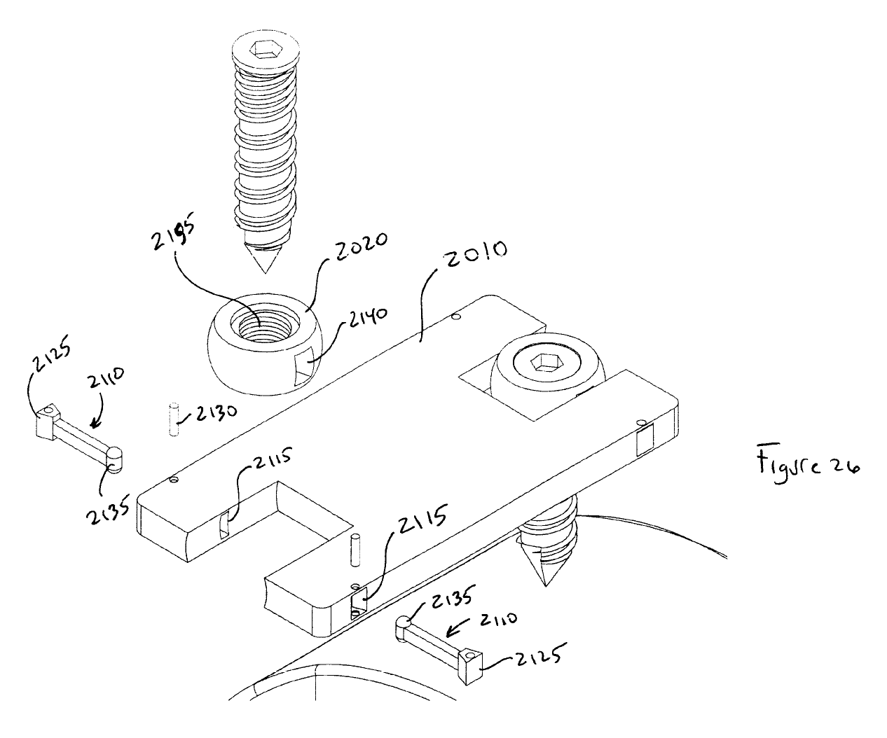

FIG. 21 shows a perspective view of another hybrid device 2105 that features a fixation device at the fusion level and an articulating surface placed within the disc space at the mobile level. The device is shown in an assembled state and attached to vertebrae V1, V2 and V3. The device fixates vertebrae V2 and V3 relative to one another so as to promote fusion while articulating surface 2205 permits movement of the vertebra V1 relative to the vertebrae V2 and V3. FIG. 22 shows an exploded view of the device of FIG. 21. The device 2105 includes a plate member 2110 that attaches to the vertebrae V2 and V3. In this regard, the plate member 2110 includes boreholes that couple to at least one bone screw per vertebral level. The inferior boreholes that overlie vertebra V3 have a largely spherical configuration and a bottom aperture that have a diameter greater than that of the bone screws. Because of the size differential between the bottom aperture of the borehole and the diameter of the screw, the spherical head of the screw can rotate within the borehole. This mechanism allows the unhindered movement of vertebra V3 towards vertebra V2. Conversely, the superior boreholes that overlie vertebra V2 have a spherical inner surface and a bottom aperture with a diameter minimally larger than that of the bone screws so that, once seated, the screws are constrained and immobile relative to the device. The plate member 2110 can have various structural shapes and configurations. For example, the plate member 2110 can include a central aperture that provides access to the disc space between the vertebrae V2 and V3. The plate member 2110 can also include a slot that provides a location where a distraction screw can be attached to the underlying vertebra.

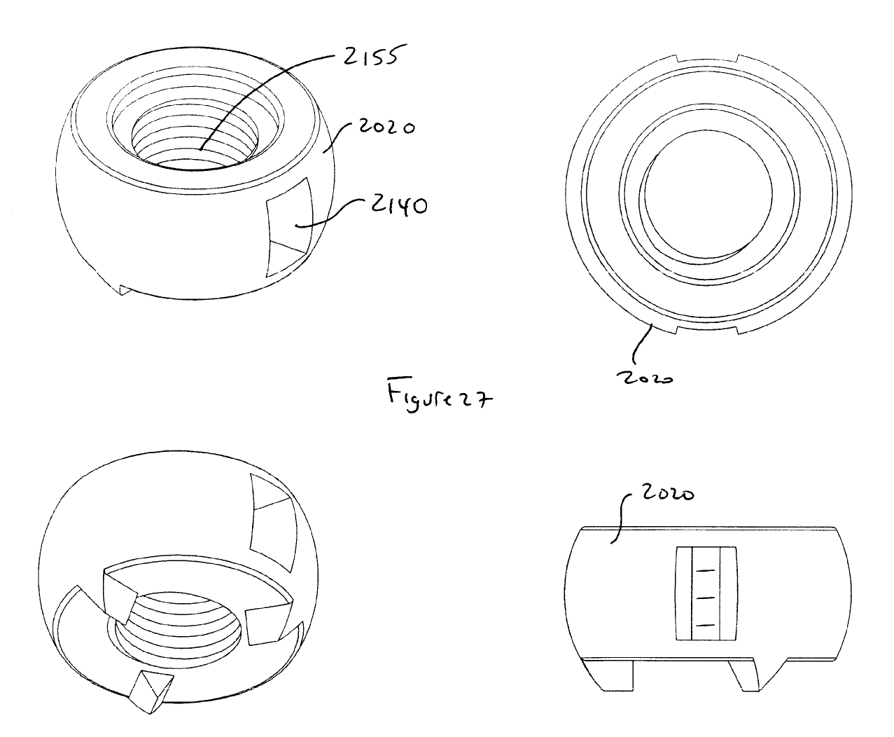

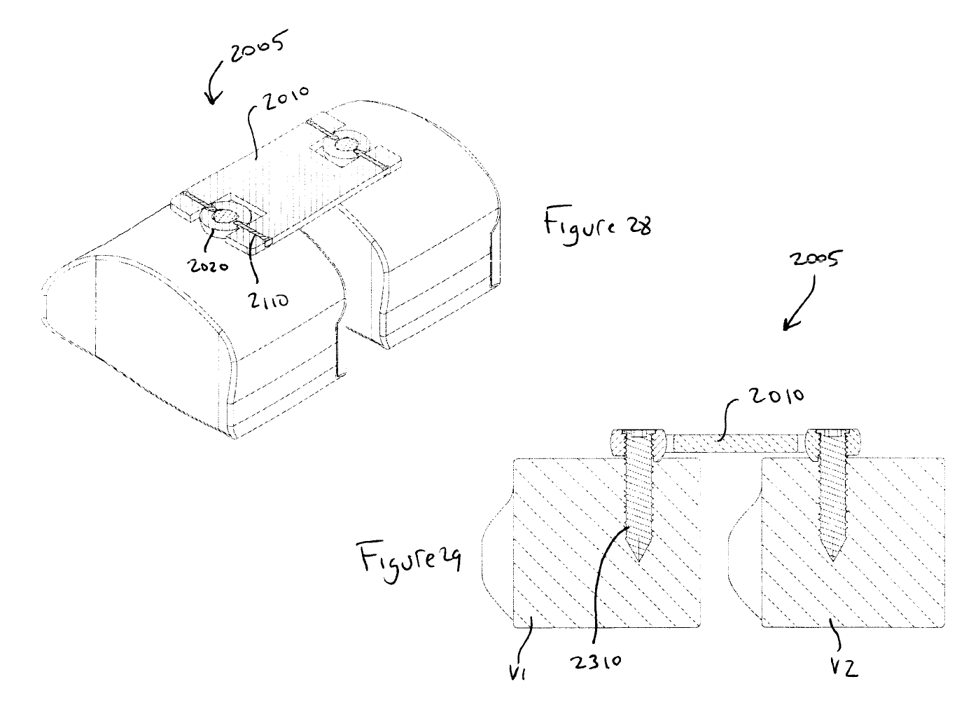

With reference to FIG. 22, an articulation member 2205 extends from one end of the plate member 2110. The articulation member 2205 is sized and shaped to extend into the disc space between the vertebrae V1 and V2. The articulation member 2205 has a dome-shaped bearing surface that rests against the inferior aspect of vertebra V1 and permits vertebra V1 to move relative to vertebra V2. FIGS. 23 and 24 show the device 2105 with different embodiments of the plate member 2110 that fixates vertebrae V2 and V3. These alternative members are similar to those illustrated in FIGS. 4 and 5.

FIG. 25 shows a perspective view of an embodiment of a dynamic fixation device 2005 in an assembled state and attached to a pair of vertebrae V1 and V2. FIG. 26 shows a perspective view of the device of FIG. 25 in an exploded state. With reference to FIGS. 25 and 26, the device 2005 includes a member 2010 that is sized and shaped to extend between the two vertebrae V1 and V2. Member 2010 includes a pair of openings 2015 that are each movably coupled to a bone screw receiver 2020 and sized and shaped to receive the screw receiver 2020. The receiver 2020 is coupled to the member 2010 in a manner that permits relative movement between the receiver 2020 and the plate member 2010 and thereby permit movement between the plate member 2010 and the screw.

FIG. 27 shows various views of the bone screw receiver 2020. With reference to FIGS. 26 and 27, receiver 2020 has a spherical outer wall that is configured to fit within the complementary spherical walls 2017 of opening 2015 of the member 2010. Each receiver 2020 includes a central bore 2155 that is threaded for receipt of a correspondingly-threaded shank of a bone screw. One or more malleable couplers 2110 are adapted to secure the receiver 2020 to the member 2010. Each malleable coupler 2110 is an elongate member that fits through a bore 2115 in the member 2010. The couplers 2110 have a first end 2125 that attaches to the member 2010 via an attachment pin 2130. A second end 2135 of the coupler 2110 fits within a receptor hole 2140 in the receiver 2020 to movably :secure the receiver 2020 to the member 2010.

FIGS. 28 and 29 show cross-sectional views of the device 2005. As shown in FIG. 29, the ends of the couplers 2110 extend into the receivers 2020 to thereby secure the receivers 2020 to the member 2010. The couplers 2110 are adapted to flex or otherwise articulate to permit relative movement between the receivers 2020 and the member 2010. The interaction of the spherical outer wall of receiver 2020 with complementary spherical walls 2017 determines the overall motion trajectory of receiver 2020 relative to member 2010. As shown in FIG. 29, the bone screws 2310 extend through the receivers 2020 into the vertebrae V1 and V2. Because the receivers 2020 can move relative to the member 2010, the bone screws 2310 can also move relative to the member 2010 while remaining attached to the plate 2010.

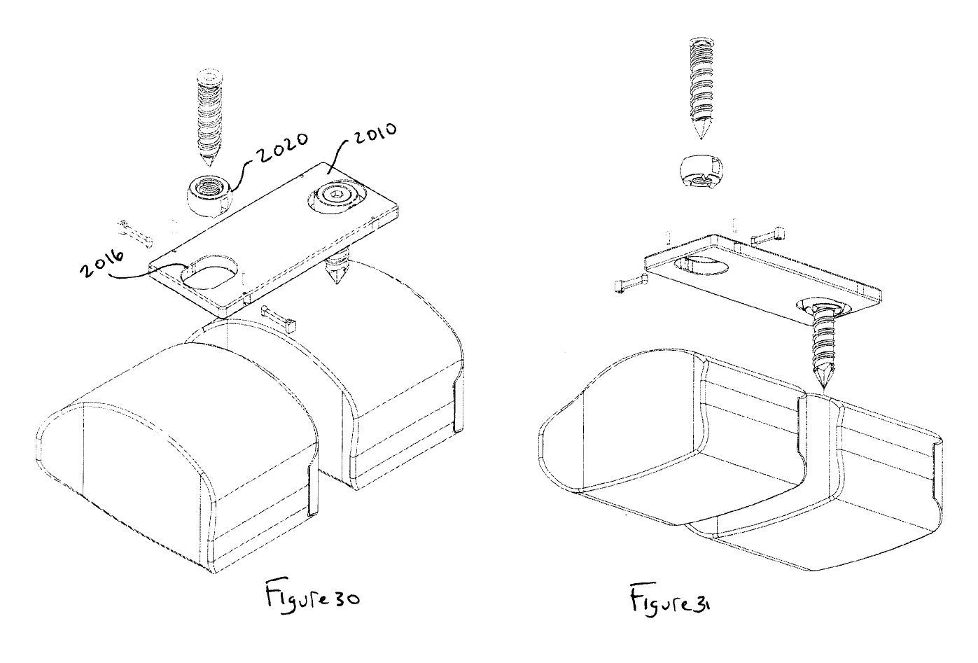

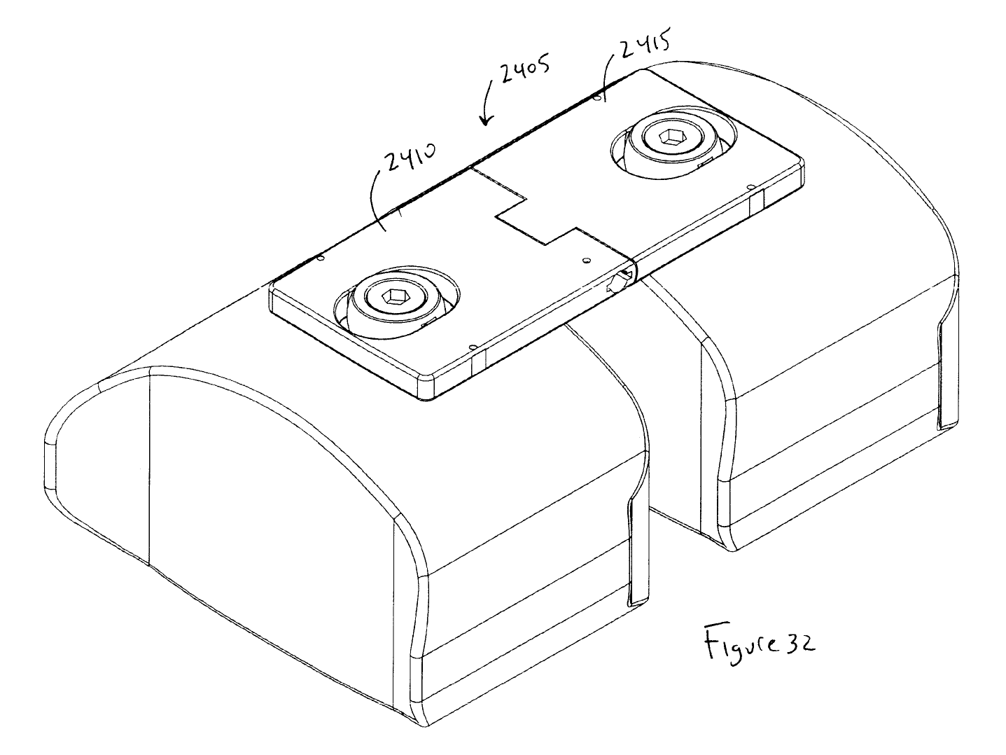

FIGS. 30 and 31 show another embodiment of the device 2005 of FIGS. 25-29. This embodiment is similar to the previous embodiment except the receivers 2020 are positioned within holes 2016 in the member 2010 rather than within open-ended slots 2015.

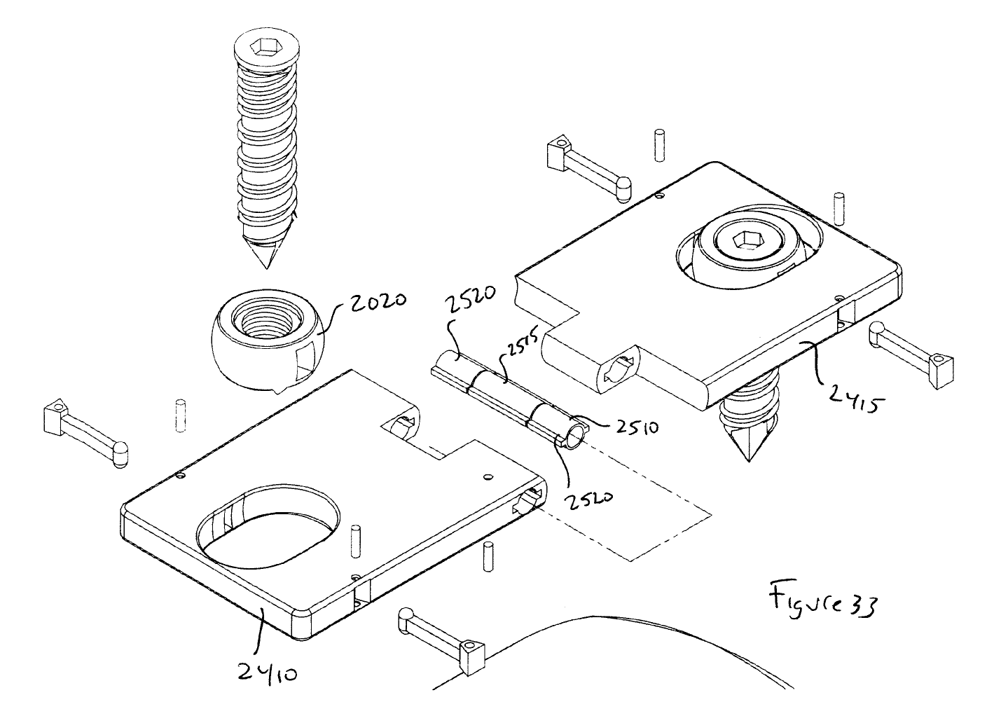

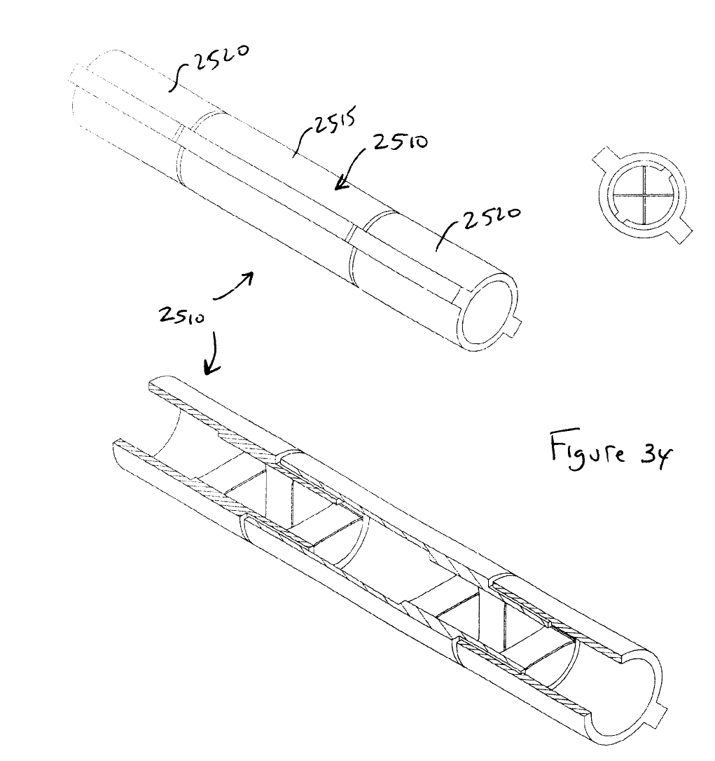

FIG. 32 shows a perspective, assembled view of another embodiment of a dynamic bone fixation device 2405. FIG. 33 shows an exploded view of the device of FIG. 32. The device 2405 has a configuration that is substantially similar to the embodiment of FIGS. 30-31. However, the plate member includes two plate components 2410 and 2415 that are movably attached to one another, as described below. The receivers 2020 are positioned within holes in the plate components and are movably attached to the plate components in the manner described with respect to the previous embodiment.

FIG. 34 shows various views of an articulating member 2510 that movably links the plate components of the device. With reference to FIGS. 33 and 34, the plate components 2410 and 2415 are connected to one another via the articulating member 2510, which is positioned within boreholes in the plate components. The articulating member 2510 is formed of a plurality of sections. The articulating member is a flexure based bearing, utilizing internal flat crossed springs, capsuled in a cylindrical housing, to provide precise rotation with low hysteresis and little frictional losses. The bearing is relatively friction-free, requires no lubrication, and is self-returning. The articulating member can resist rotational movement away from a neutral state and the extent of resistance to rotation is directly related to the extent of rotation. The extent of resistance to rotation can be a pre-determined property of the device. In one embodiment, the articulation member has high radial stiffness, high axial stiffness and is frictionless (hence, no particle wear debris). An exemplary articulating member 2510 of the type shown in FIGS. 33 and 34 is distributed by Riverhawk Company of New York under the name FREE FLEX PIVOT.

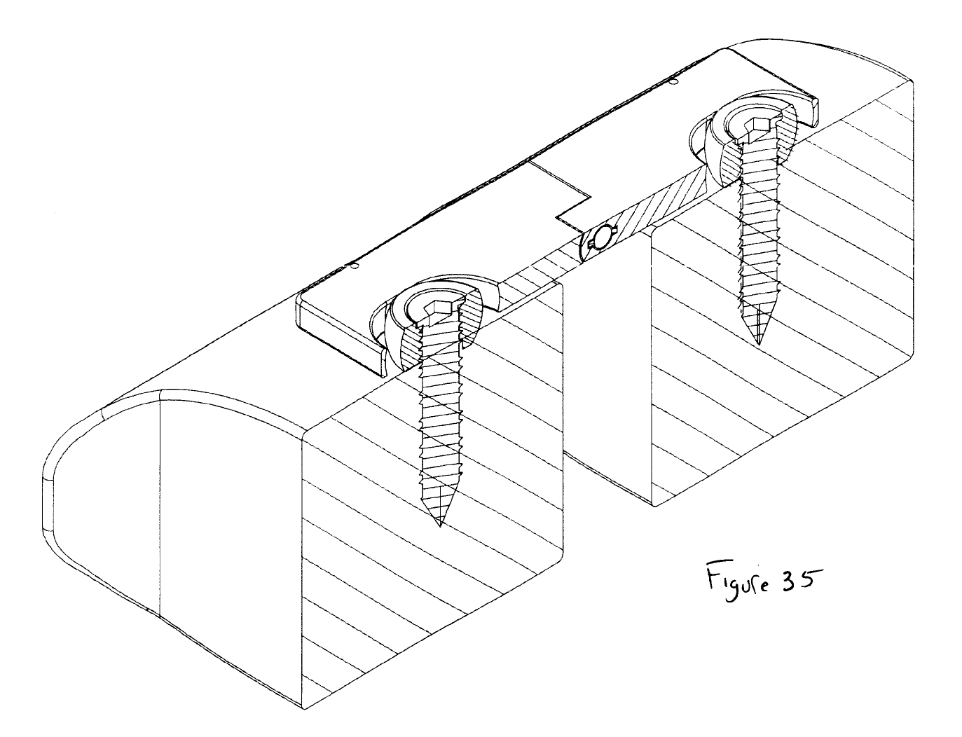

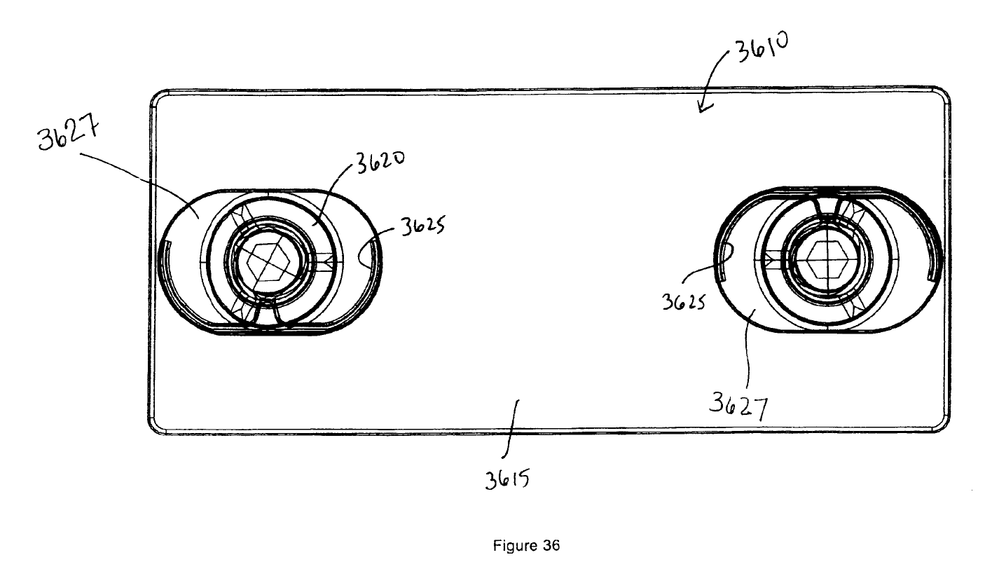

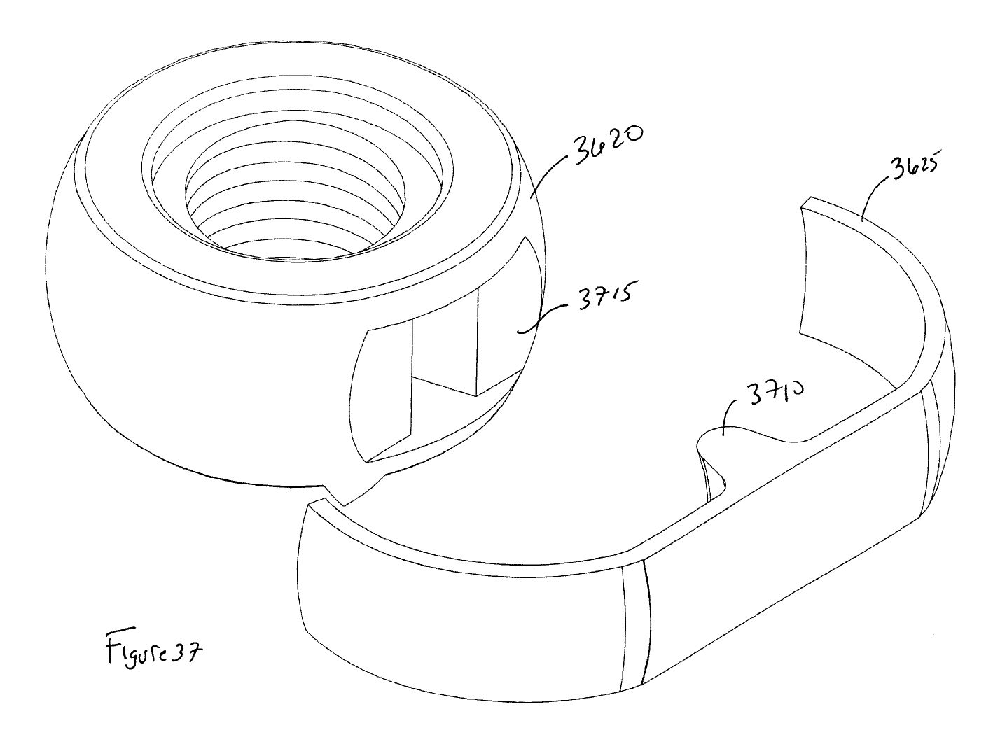

The articulating member 2510 includes a first portion 2515 that is positioned inside the plate component 2415 while the hinge portions 2520 are positioned inside the plate component 2410. In this manner, the components 2415 and 2410 can rotate relative to one another via the articulating member 2510. FIG. 35 shows a cross-sectional view of the device of FIG. 32 attached to the vertebrae.

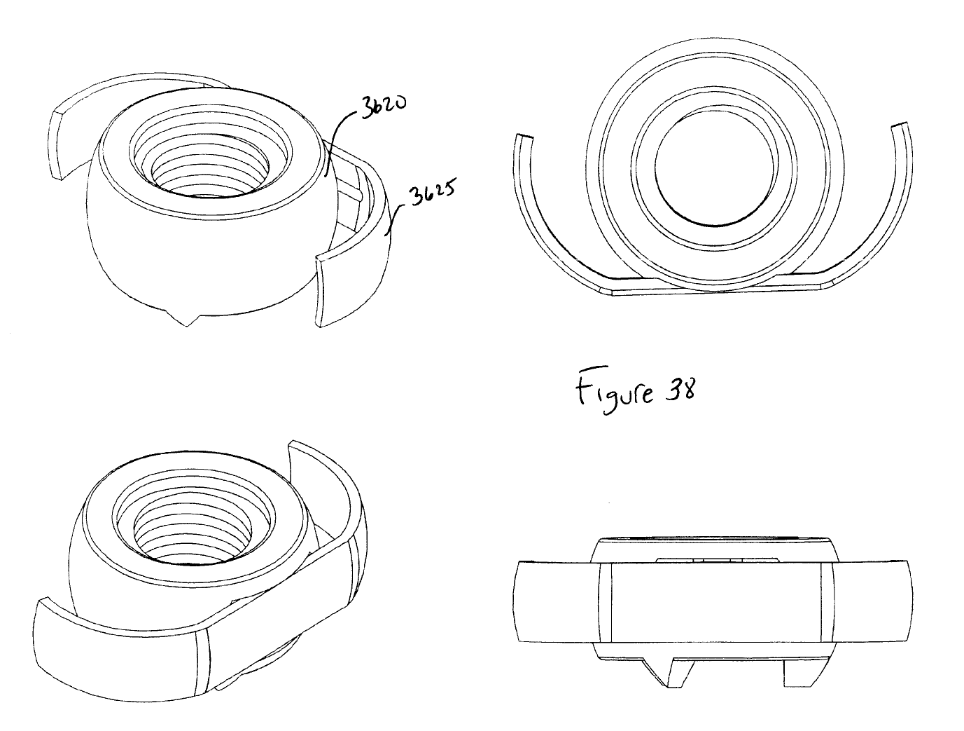

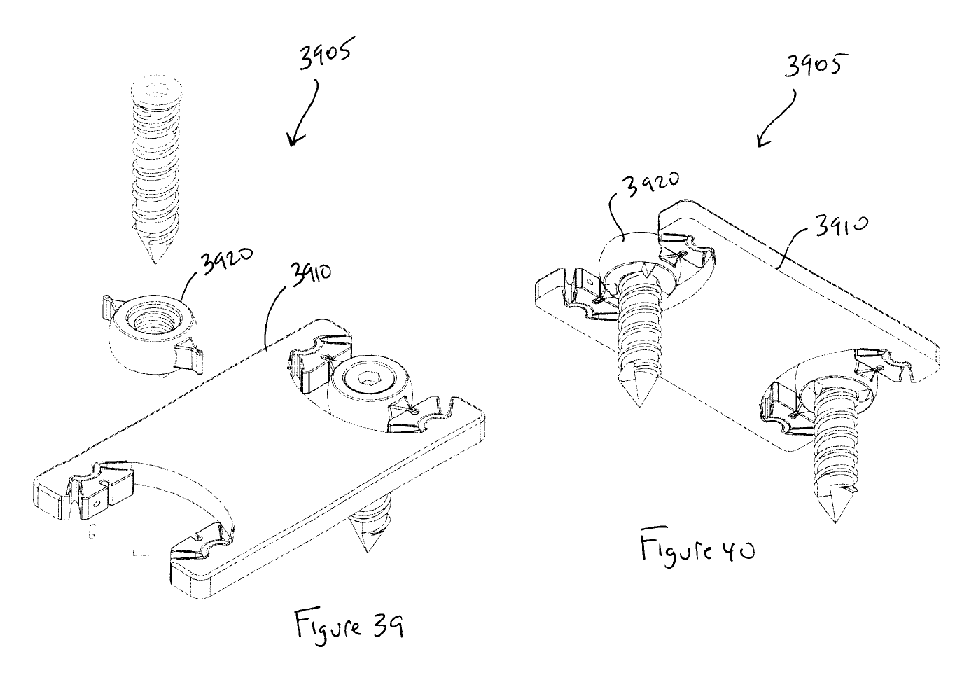

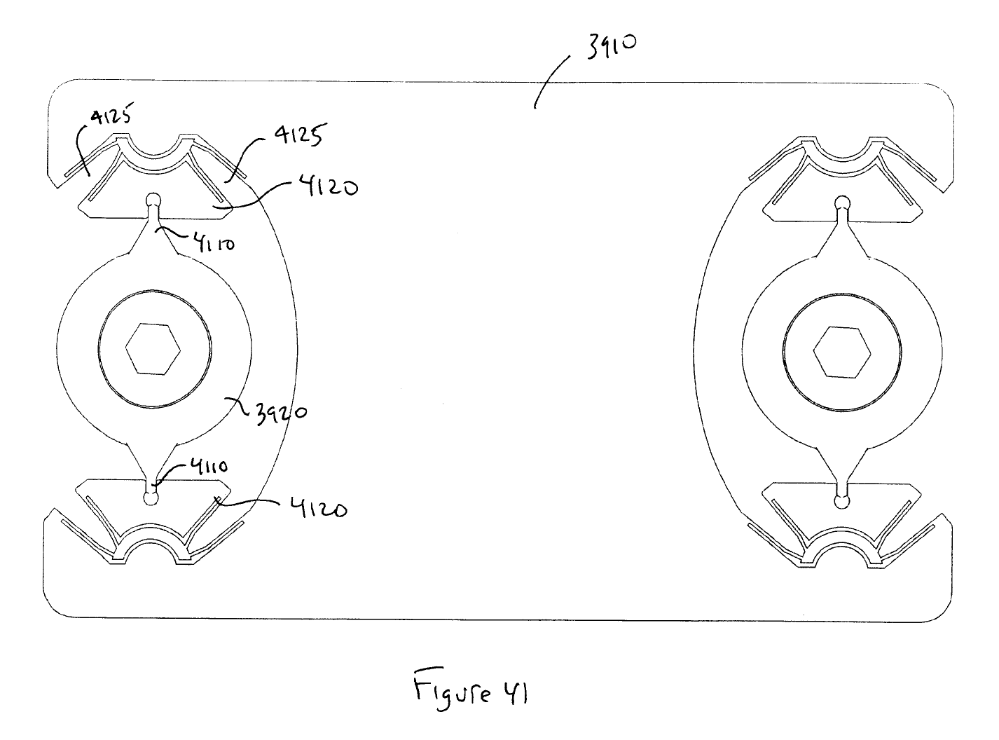

FIG. 36 shows another embodiment of a bone fixation device 3610. The device 3610 includes a plate member 3615 having bone screw receivers 3620 that movably reside within elongated slot 3627. Movement of receiver 3620 within slot 3627 is resisted by malleable member 3625 so that the receiver 3620 may move from a first position to a second position relative to slot 3627 in reaction to an applied force and then substantially return to the first position when the force has dissipated. FIG. 37 shows an enlarged view of a receiver 3620 and malleable member 3625. Member 3625 includes a knob 3710 that fits within a complementary-shaped hole 3715 in the receiver 3620. While not depicted for diagrammatic simplicity, a pin or small screw may be driven from the top surface of receiver 3620, through hole 3715 and the knob 3710 retained within, and into a portion of the bottom surface of the receiver 3620 in order to more rigidly affix malleable member 3625 to receiver 3620. FIG. 38 shows various views of the coupler 3625 and receiver 3620 in an assembled state. Bone fixation spikes (or other texturing or protrusions) can be situated along the inferior, bone-contacting surface of receiver 3620 in order to increase the extent of bone contact and fixation. The spherical outer walls of receiver 3620 are adapted to fit within slot 3627 and provide a bearing surface with the complementary spherical inner walls of slot 3627.

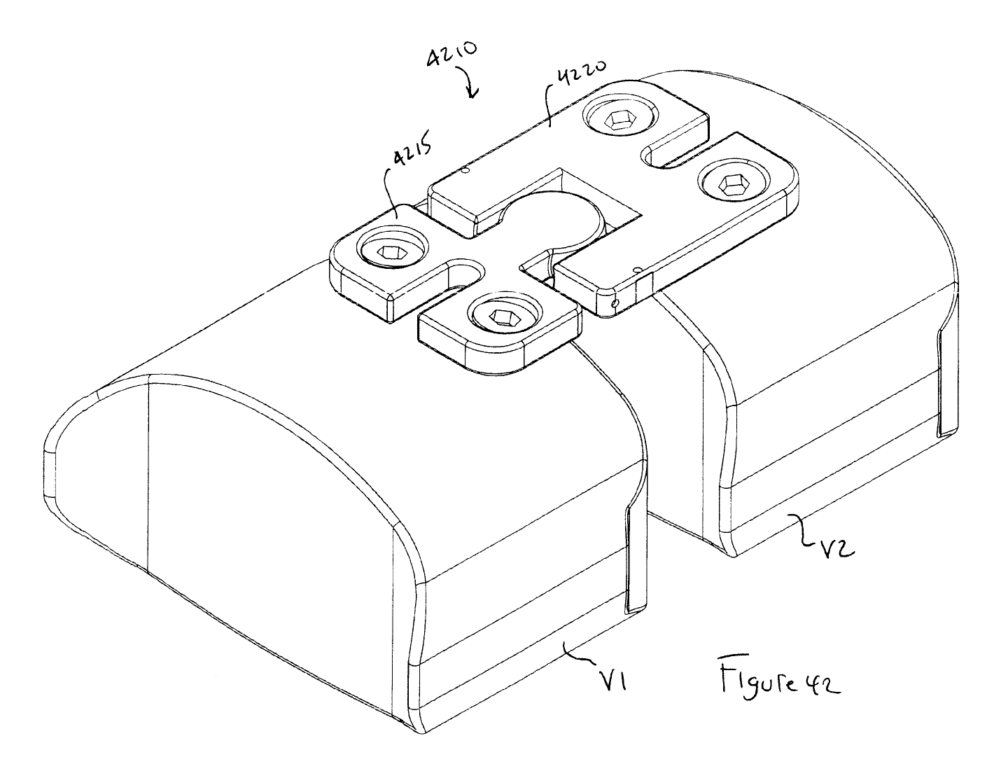

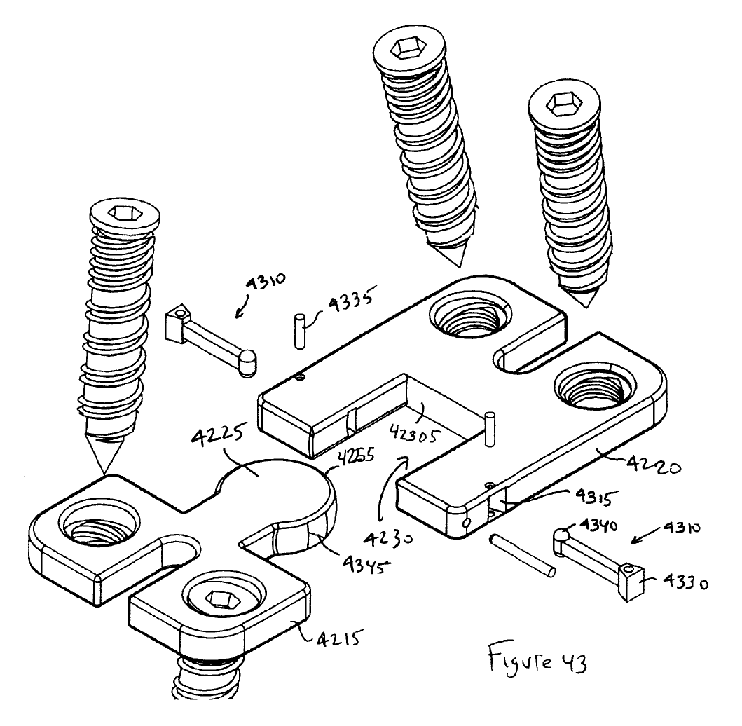

FIGS. 39 and 40 show top and bottom perspective views of another embodiment of a device 3905. FIG. 41 shows a top view of the device of FIG. 39. The device 3905 includes a plate member 3910 that movably couples to at least one bone screw receiver 3920. The bone screw receiver 3920 has an internal bore that is sized to receive a shank portion of a bone screw. The bore is threaded to mate with corresponding threads in the screw. With reference to FIG. 41, the receiver 3920 has a pair of pin-shaped protrusion 4110 that mate with corresponding couplers 4120 of the malleable assemblies 4130 of the plate 3910. The couplers 4120 are fan-shaped and are adapted to move relative to the plate such as within spaces 4125 on either side of the couplers 4120. This permits the couplers 4120 to move or articulate relative to the remainder of the plate such as in response to forces exerted on or by the attached vertebrae. In this way, movement of each receiver 3920 relative to plate 3910 is resisted by each malleable assembly 4130 so that the receiver 3920 may move from a first position to a second position in reaction to an applied force and then substantially return to the first position when the force has dissipated.

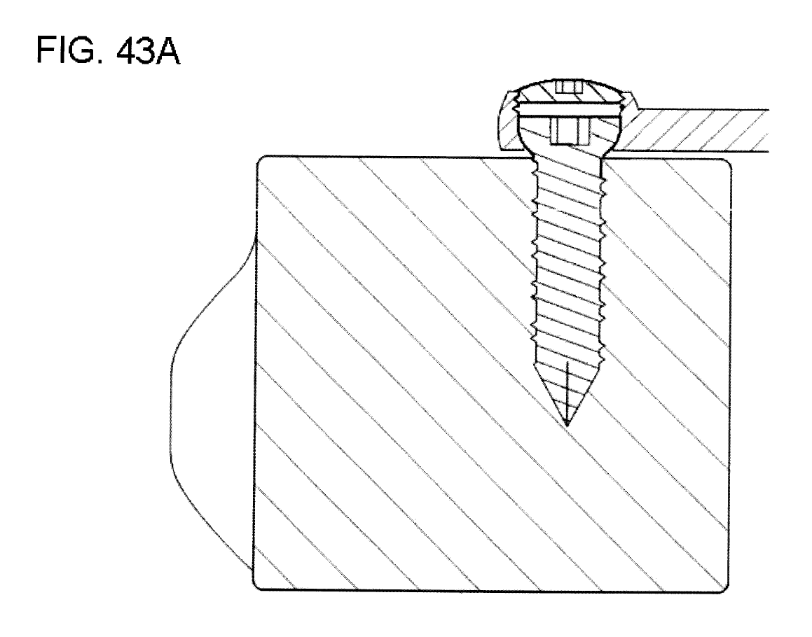

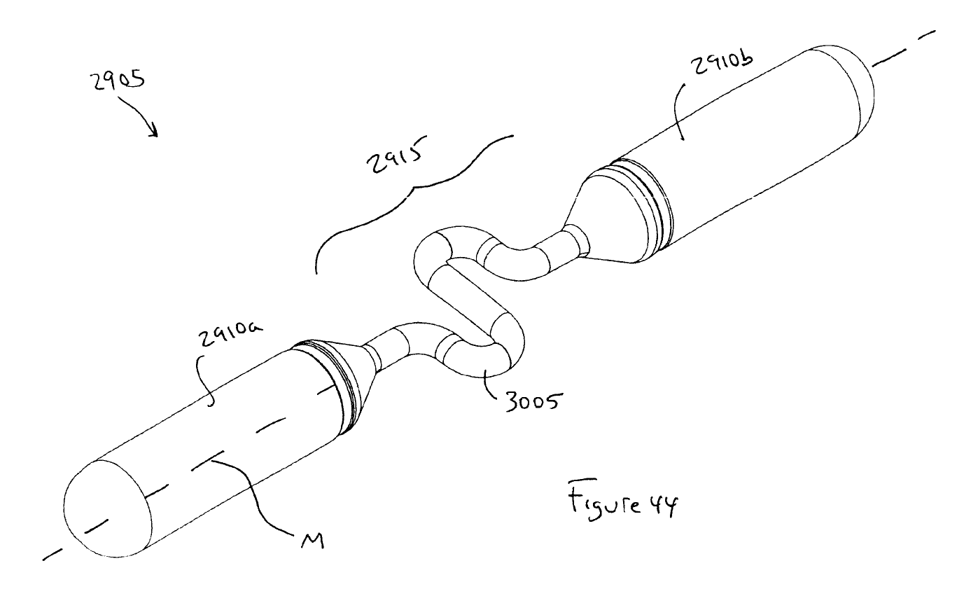

FIG. 42 shows yet another embodiment of a bone fixation device 4210. FIG. 43 shows an exploded view of the device of FIG. 42. The device 4210 includes two components members 4215 and 4220 that are movably attached to one another, as described below. Each component member includes one or more boreholes for receipt of bone fasteners. The component member 4215 includes a protrusion 4225 that is positioned within a slot 4230 in the plate component 4220. Protrusion 4225 can have a partially circumferential convex spherical wall 42255 that interacts with complementary concave spherical walls 42305 of slot 4230.

With reference to FIG. 43, one or more malleable couplers 4310 are adapted to secure the protrusion 4225 to the component 4220. Each coupler 4310 is an elongate, flexible member that fits through a bore 4315 in the plate component 4220. The couplers 4310 have a first end 4330 that attaches to the plate member 2010 via an attachment pin 4335. A second end 4340 of the coupler fits within a receptor hole 4345 in the protrusion 4225 to secure the protrusion to the plate component 4220. The movement of one component member 4215 relative to the other component member 4220 is resisted by each flexible malleable coupler 4310 so that one component member may move from a first position to a second position relative to the other component member in reaction to an applied force and then substantially return to the first position when the force has dissipated.

The embodiments illustrated in FIGS. 25 to 43 disclose various devices that can be used to support and maintain movement at the non-fused disc level. Removal of the malleable members from any of these devices would permit unhindered subsidence at the fixated level and render the respective embodiment suitable for use at the fused level. (Complete immobilization of the device components (with or without removal of the malleable member) would also produce a device that can be used at the fused levels. However, these rigid devices are less preferable than those that would accommodate boney subsidence.) A hybrid device that employs one or more of the device embodiments illustrated in FIG. 25 to 43 and one or more of a fusion device can be made to simultaneously accommodate both fusion and dynamic movement at different disc levels. While a number of fusion devices have been disclosed and illustrated in this application, it is understood that any of the numerous fusion fixation devices that are currently known in the art may be alternatively used with the malleable embodiments to produce the desired hybrid fixation devices. In addition to the illustrated plate fixation devices, known fixation devices include rid-based embodiments such as those disclosed in U.S. Pat. Nos. 5,800,433; 5,713,900, Pub. No. 2005/0004573 and others. Further, embodiments that can support and maintain movement at the non-fused disc level may be generated from the rod-based devices by the addition of a malleable member that would oppose movement of the device components away from a pre-set neutral position and then substantially return the device to that neutral position upon dissipation of the motive force. Lastly, a malleable member can be made to act directly at the bone screw/borehole interface. In FIG. 43a, the borehole has a largely spherical configuration and a bottom aperture that has a diameter greater than that of the bone screws. Because of the size differential between the bottom aperture of the borehole and the diameter of the screw, the spherical head of the screw can rotate within the borehole. A cap is threaded onto the top opening of the borehole so that a space is formed between the bottom of the cap and the top of the bone screw. The formed space can contain an elastic material(s), fluids, spring device(s), Belleville washers, magnets or any other appropriate materials/devices that will resist movement between the head of bone screw and the bottom of the cap. The material/device within the space will apply a force to the head of the screw and resist any bone screw movement away from the neutral position. In this way, the screw will move within the borehole in response to a deflecting force and will return to the neutral position when the applied force has dissipated.

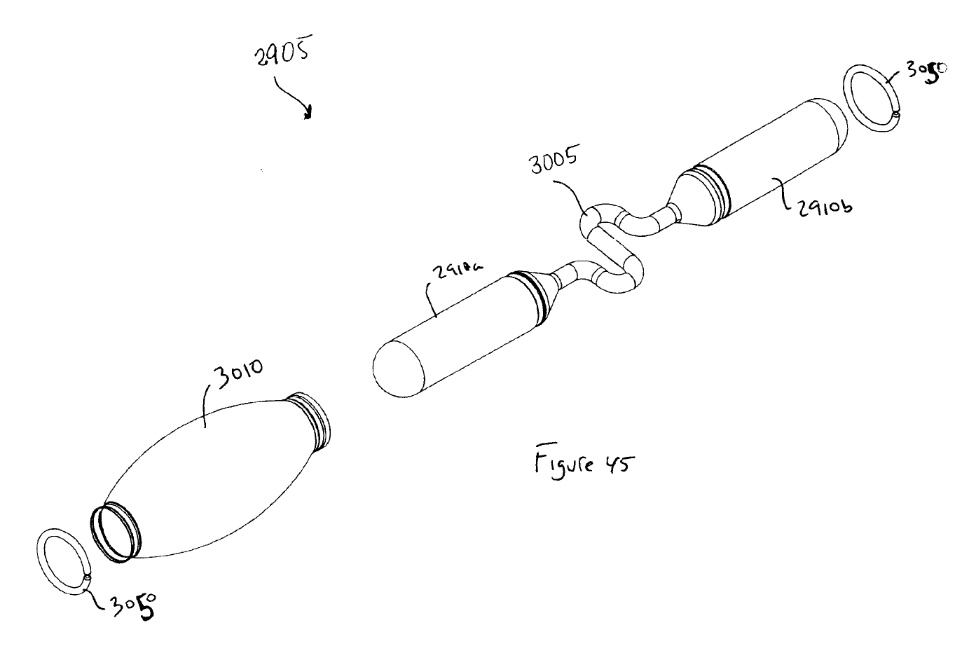

FIG. 44 is a perspective view of a dynamic bone fixation device 2905 configured to retain bone portions such as vertebra of a spinal column in a desired spatial relationship. FIG. 45 shows the device of FIG. 44 in an exploded state. With reference to FIGS. 44 and 45, the device 2905 includes first and second connection portions 2910a and 2910b that are adapted to attach to respective vertebrae. In the illustrated embodiment, the connection portions 2910 are elongated and rod-like and are adapted to attach to bone screws, as described in detail below. The device 2905 also includes a malleable portion 2915 that malleably links the connection portions 2910. The malleable portion 2915 includes a malleable member 3005 that is covered by a sheath 3010 (FIG. 30). The malleable member 3005 and sheath 3010 are adapted to alter in shape so as to permit at least limited relative displacement between the vertebrae.

With reference still to FIG. 44, the malleable member 3005 is an undulated rod that loops back and forth about a longitudinal axis of the device. The undulating shape permits the malleable member flex, move or otherwise change shape along a direction parallel to the midline M and/or a direction transverse or cross-wise to the midline M. The device 2905 can include clamps, such as c-clamps 3050 that encircle the attachment regions edges of the sheath 3010 and exert a compressive force on the sheath to removably fix the sheath 3010 to the connection portions 2910. Other structures or mechanisms can be used to attach the sheath to the device 105.

In the assembled state, the sheath 3010 encloses or encapsulates the malleable member 3005. The sheath 3010 is sized relative to the malleable member 3005 such that there is sufficient space to permit movement of the malleable member 3005 inside the sheath 3010. The sheath 3010 functions to contain shed particulate debris, keep adjacent tissues and scar out of the mobile core and, if desired, permit placement of a biocompatible lubricant within the space. Should the malleable member fail and fracture, the membrane would also serve to contain the fragments and keep the device ends attached to one another.

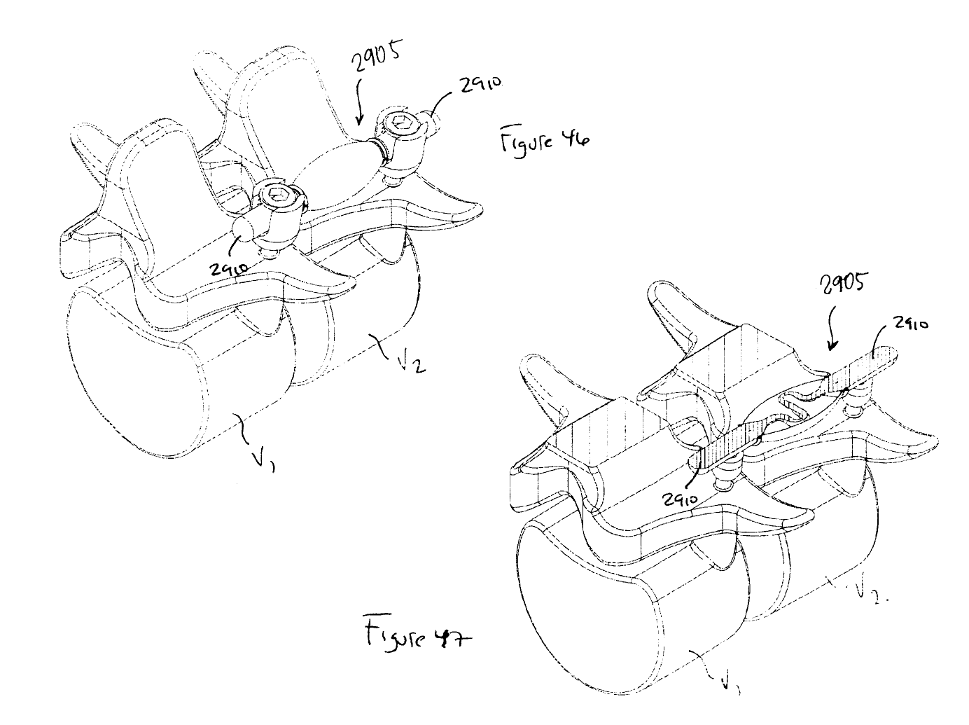

FIGS. 46 and 47 show the device 2905 of FIG. 44 attached to vertebrae V1 and V2. Each connection portion 2910 is sized and shaped to fit within a receiver of a bone screw assembly that has been attached to a vertebra. In this manner, the device 2905 interconnects the vertebrae V1 and V2 with the malleable portion permitting movement therebetween.

While many of the disclosed embodiments featured a specific screw/borehole configuration (such as the threaded screw and threaded bore hole), it should be appreciated that these configurations are exemplary and do not limit the scope of the invention. Numerous screw/borehole configurations and screw-to-borehole locking mechanisms are well known in the art and any of these may be alternatively employed to fasten the disclosed devices onto the underlying bone.

The disclosed devices or any of their components can be made of any biologically adaptable or compatible materials. Materials considered acceptable for biological implantation are well known and include, but are not limited to, stainless steel, titanium, tantalum, combination metallic alloys, various plastics, resins, ceramics, biologically absorbable materials and the like. Any components may be also coated/made with osteo-conductive (such as demineralized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation. Further, any surface may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. Lastly, the system or any of its components can also be entirely or partially made of deformable materials.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

What is claimed is:

1. A device for stabilization of a spinal segment that includes at least three vertebral bodies and two intervening discs, comprising:
a first component attachable to a first vertebral body;
a second component attachable to a second vertebral body and movably connected to the first component across a first disc, wherein the first component and second component are adapted to reversibly move from a first relative position to a second relative position in reaction to an applied force that produces vertebral motion and are further adapted to substantially return to the first relative position upon dissipation of the applied force in response to a force produced by the device so as to support vertebral motion across the first disc; and
a third component attachable to a third vertebral body and connected to the second component across a second disc, wherein the second component and third component are adapted to move from a first relative position to a second relative position in reaction to an applied force that produces vertebral motion but, upon dissipation of the initial applied force, the device produces no motive force onto the vertebral bodies and there is no device-generated vertebral motion so as to accommodate fusion across the second disc.

2. A device as in claim 1, wherein at least one bone anchor attaches each component to a respective vertebral body.

3. A device as in claim 1, further comprising an articulating component connecting the first component to the second component.

4. A device as in claim 3, further comprising a sheath that encloses the articulating component.

5. A device as in claim 1, wherein the second component and the first component are both part of a plate.

6. A device for stabilization of a spinal segment that includes at least three vertebral bodies and two intervening discs, comprising:
a first component attachable to a first vertebral body;
a second component attachable to a second vertebral body and movably connected to the first component across a first disc, wherein the first component and second component are adapted to reversibly move from a first relative position to a second relative position in reaction to an applied force that produces vertebral motion and are further adapted to substantially return to the first relative position upon dissipation of the applied force secondary to the action of a force produced by the device so as to support vertebral motion across a first disc; and
a third component attachable to a third vertebral body and connected to the second component across a second disc, wherein the second component and third component are adapted to promote fusion by rigidly affixing the second and third vertebral bodies.

7. A device for spinal stabilization of at least two vertebral bodies that are adapted to rotate relative to one another about a radius defined by a center of rotation, comprising:
a first component attachable to a first vertebral body;
a second component attachable to a second vertebral body; and
at least one pair of complementary bearing surfaces connecting the first and second components across a first disc, the bearing surfaces adapted to movably interact upon a curvilinear path defined by the radius centered about a point of rotation.

8. A device as in claim 7, wherein the center of rotation corresponds to the physiological instantaneous axis of rotation of the vertebral bodies.

9. A device as in claim 7, wherein the center of rotation is stationary.

10. A device as in claim 7, wherein the center of rotation is mobile.

11. A device as in claim 7, further comprising a third component attachable to a third vertebral body and connected to the second component across a third disc.

12. A device as in claim 7, wherein the first component and second component are adapted to reversibly move from a first relative position to a second relative position in reaction to an applied force that produces vertebral motion and are further adapted to substantially return to the first relative position upon dissipation of the applied force secondary to the action of a force produced by the device so as to support vertebral motion across the first disc.

13. A device as in claim 11, wherein the second component and third component are adapted to move from a first relative position to a second relative position in reaction to an applied force that produces vertebral motion but, upon dissipation of the initial applied force, the device produces no motive force onto the vertebral bodies and there is no device-generated vertebral motion so as to accommodate fusion across the second disc.

14. A device as in claim 7, further comprising an additional bearing surface positioned within the first disc, wherein the additional bearing surface is defined by the radius.

15. A device for spinal stabilization, comprising:
a first component that anchors to a first vertebral body;
a second component that anchors to a second vertebral body; and
an intervening member adapted to provide interaction between the first and second components without a direct bearing surface between the first and second components, wherein the intervening member contains at least one bearing surface that is adapted to articulate with at least one of the components anchored to a vertebral body and further comprising at least one bearing surface between the intervening member and an anchored component that is adapted to reversibly move from a first relative position to a second relative position in reaction to an applied force that produces vertebral motion and is further adapted to substantially return to the first relative position upon dissipation of the applied force secondary in response to a force produced by the device so as to support vertebral motion across a disc space.

16. A device as in claim 15, further comprising a third component attachable to a third vertebral body and connected to the second component across a second disc, wherein the second component and third component are adapted to move from a first relative position to a second relative position in reaction to an applied force that produces vertebral motion but, upon dissipation of the initial applied force, the device produces no motive force onto the vertebral bodies and there is no device-generated vertebral motion so as to accommodate fusion across the second disc.

17. A device as in claim 15, further comprising a third component attachable to a third vertebral body and connected to the second component across a second disc, wherein the second component and third component are adapted to promote fusion by rigidly affixing the second and third vertebral bodies.

18. A device for stabilization of a spinal segment, comprising:
a first component attachable to a first vertebral body;
a second component attachable to a second vertebral body;
a malleable member connecting the first and second components, the malleable member adapted to provide movement between the first and second components from a first position to a second position in reaction to an applied force producing vertebral motion and to substantially return the first and second components to the first position when force of motion has dissipated; and
a biocompatible membrane that at least partially surrounds the malleable member so as to at least partially separate the malleable member from the surrounding environment when the device is attached to the spinal segment;
wherein the device does not reside within a disc space but is anchored to a side of the spinal segment using a plurality of bone fasteners.

19. A device as in claim 18, wherein each component anchors to a respective vertebral body using at least one bone anchor.

20. A device as in claim 18, wherein the malleable member is an undulating rod.

21. A device as in claim 18, further comprising a third component attachable to a third vertebral body and connected to the second component across a second disc, wherein the second component and third component are adapted to move from a first relative position to a second relative position in reaction to an applied force that produces vertebral motion but, upon dissipation of the initial applied force, the device produces no motive force onto the vertebral bodies and there is no device-generated vertebral motion so as to accommodate fusion across a second disc.

22. A device as in claim 18, further comprising a third component attachable to a third vertebral body and connected to the second component across a second disc, wherein the second component and third component are adapted to promote fusion by rigidly affixing the second and third vertebral bodies.

23. A device for stabilization of a spinal segment that includes at least three vertebral bodies and two intervening discs, comprising:
a first component attachable to a first vertebral body;
a second component attachable to a second vertebral body and connected to the first component across a first disc, wherein the first component and second component are adapted to move toward one another from a first position to a second position in reaction to an applied force producing vertebral motion of the first vertebral body toward the second vertebral body but not to return to the first position upon dissipation of the applied force so as to promote spinal fusion across the first disc; and
at least one bearing component connected to the second component, the bearing component adapted to extend into a second disc between the second vertebral body and a third vertebral body, the bearing component adapted to support defined vertebral motion across the second disc.

24. A device as in claim 23, wherein the vertebral motion across the second disc is curvilinear motion.

25. A device as in claim 23, wherein the bearing component has a domed bearing surface.

26. A device for dynamic spinal stabilization of at least two vertebral bodies, comprising:
a first component attachable to an anterior surface of a first vertebral body;
a second component attachable to an anterior surface of a second vertebral body; and
at least one complementary bearing surface between the first and second components, wherein the bearing surface is adapted to provide movement between the two attachable components from a first relative position to a second relative position in reaction to an applied force that produces vertebral motion and is further adapted to substantially return the two attachable components to the first relative position upon dissipation of the applied force secondary to the action of a force produced by the device.

27. A device as in claim 26, further comprising a third component attachable to a third vertebral body and connected to the second component across a second disc, wherein the second component and third component are adapted to move from a first relative position to a second relative position in reaction to an applied force that produces vertebral motion but, upon dissipation of the initial applied force, the device produces no motive force onto the vertebral bodies and there is no device-generated vertebral motion so as to accommodate fusion across a second disc.

28. A device as in claim 26, further comprising a third component attachable to a third vertebral body and connected to the second component across a second disc, wherein the second component and third component are adapted to promote fusion by rigidly affixing the second and third vertebral bodies.

29. A device for stabilization of a spinal segment that includes at least three vertebral bodies and two intervening discs, comprising:
a first component attachable to a first vertebral body;
a second component attachable to a second vertebral body and movably connected to the first component across a first disc, wherein the first component and second component are adapted to reversibly move from a first relative position to a second relative position in reaction to an applied force that produces vertebral motion and are further adapted to substantially return to the first relative position upon dissipation of the applied force secondary to the action of a force produced by the device so as to support vertebral motion across a first disc; and
a fourth component attachable to a third vertebral body and connected to a third component across a second disc, wherein the second component and third component are modularly attached to one another and wherein the third and fourth components are further adapted to move from a first relative position to a second relative position in reaction to an applied force that produces vertebral motion but, upon dissipation of the initial applied force, the device produces no motive force onto the vertebral bodies and there is no device-generated vertebral motion so as to accommodate fusion across a second disc.

30. A device for stabilization of a spinal segment that includes at least three vertebral bodies and two intervening discs, comprising:
a first component attachable to a first vertebral body;
a second component attachable to a second vertebral body and movably connected to the first component across a first disc, wherein the first component and second component are adapted to reversibly move from a first relative position to a second relative position in reaction to an applied force that produces vertebral motion and are further adapted to substantially return to the first relative position upon dissipation of the applied force secondary to the action of a force produced by the device so as to support vertebral motion across a first disc; and
a fourth component attachable to a third vertebral body and connected to a third component across a second disc, wherein the second component and third component are modularly attached to one another and wherein the third and fourth components are further adapted to promote fusion by rigidly affixing the second and third vertebral bodies.

31. A method for stabilization of a spinal segment that includes at least three vertebral bodies and two intervening discs, comprising:
attaching a first component to a first vertebral body;
attaching a second component to a second vertebral body such that the second component is movably connected to the first component across a first disc, wherein the first component and second component are adapted to reversibly move from a first relative position to a second relative position in reaction to an applied force that produces vertebral motion and are further adapted to substantially return to the first relative position upon dissipation of the applied force secondary to the action of a force produced by the device so as to support vertebral motion across a first disc; and
attaching a third component to a third vertebral body such that the third component is connected to the second component across a second disc, wherein the second component and third component are adapted to move from a first relative position to a second relative position in reaction to an applied force that produces vertebral motion but, upon dissipation of the initial applied force, the device produces no motive force onto the vertebral bodies and there is no device-generated vertebral motion so as to accommodate fusion across a second disc.

32. A bearing device for the movable connection of at least two components of a medical implant, wherein:
the bearing device is adapted to reversibly rotate attached components about a central axis of rotation from a first relative position to a second relative position in reaction to an applied motive force and to substantially return the attached components to the first relative position upon dissipation of the applied force secondary to the action of a force produced by the device; and
wherein a force of restoration produced by the device is derived at least in part from radial leaf springs that at least partially occupy and transverse a hollow center of the device.
